(19) United States
(12) Patent Application Publication
(10) Pub. No.: US 2022/0184192 A1
(43) Pub. Date: Jun. 16, 2022

(54) A Composition for the Delivery of Biologically Active Agents and Uses Thereof

(71) Applicant: Cytomatrix Limited, Melboume (AU)

(72) Inventors: Christina KIRKLAND, Waurn Ponds (AU); Alessandra SUTTI, Waurn Ponds (AU); Julie SHARP, Waurn Ponds (AU); Mark Kirkland, (US); Guy MOENECLAEY, Clayton (AU)

(21) Appl. No.: 17/599,237

(22) PCT Filed: Apr. 2, 2020
(86) PCT No.: PCT/AU2020/050327

§ 371 Date: Sep. 28, 2021

(30) Foreign Application Priority Data
Apr. 2, 2019 (AU) 2019901112

(51) Int. Cl. A61K 39/00; A61K 9/70; A61K 38/19; A61K 39/39; A61K 47/34; A61P 37/04

(52) U.S. Cl. CPC A61K 39/0011; A61K 9/70; A61K 38/193; A61K 39/39; A61K 2039/55561; A61P 37/04; A61K 2039/6093; A61K 2039/5152; A61K 47/34

(57) ABSTRACT

The invention relates generally to a composition for rapid and sustained delivery of one or more biologically active agents, and uses thereof, wherein the composition comprises short biocompatible polymer fibres (SPF) having an average length in the range of from about 1 pm to about 3 mm, and an average diameter in the range of from about 15 nm to about 5 μm, wherein the SPF are loaded with one or more biologically active agents, and wherein, when administered, the composition provides rapid and sustained release of the one or more biologically active agents from the SPF.

### TECHNICAL FIELD

The present invention relates generally to a composition for the delivery of a biologically active agent. In particular, the present invention relates to a composition comprising short biocompatible polymer fibres for the rapid and sustained delivery of one or more biologically active agent, and to uses thereof.

### BACKGROUND

All references, including any patent or patent application cited in this specification are hereby incorporated by reference to enable full understanding of the invention.

Nevertheless, such references are not to be read as constituting an admission that any of these documents forms part of the common general knowledge in the art, in Australia or in any other country.

Whilst the efficacy of biologically active agents in therapy is critically dependent upon the mechanism(s) of action of the agents used, other factors can also be important in eliciting the optimal or appropriate response. Tolerable dose and time of administration relative to onset of the disease or disorder to be treated are often key considerations. There are also a number of complex issues involving pharmacokinetic and pharmacodynamic characteristics that can also contribute to the desired therapeutic response.

Previous studies have been carried out with a vast array of therapeutic agents in order to establish optimal strategies for the delivery of active agents, including therapeutic agents. Biologically active agents may be incorporated into a number of different dosage forms or delivery vehicles for administration across different routes, with the choice of dosage form or delivery vehicle typically determined by the intended route of administration. Illustrative examples of suitable dosage forms or delivery vehicles include tablets, capsules, sprays, ointments or patches for delivery of biologically active agents by routes such as intravascular (e.g., intravenous), subcutaneous, intraperitoneal, intramuscular, oral, sublingual, transmucosal and transdermal routes of administration.

It is generally understood that many biologically active agents are not suitable for particular routes of administration. For instance, many biologically active agents are susceptible to degradation by proteolytic enzymes and/or stomach acid, or they may be insufficiently absorbed into the systemic circulation by restrictions such as molecular weight and/or charge, in particular when administered by oral, transmucosal or transdermal routes.

Many biologically active agents also require repeated administration over a period of time to achieve or maintain a desired therapeutic response. This is evident, for example, with immunotherapy, where immunisation generally requires multiple vaccinations, boosters and/or high doses of vaccine compositions to be administered, resulting in increased economic costs to patients and the healthcare sector.

These disadvantages have been partly mitigated by the use of small diameter particles that encapsulate the biologically active agent(s) and thereby protect it/them from degradation following administration. Such particles are often formed from synthetic degradable polymers that break down in a biological environment to release the encapsulated agent over a period of time.

Strategies involving the use of delivery vehicles that can encapsulate active agents in such a way as to allow for protection and controlled release have shown promise as a way of optimizing the delivery characteristics of drugs and other biologically active agents. Such vehicles offer the possibility of successful treatment and control of many diseases with agents whose systemic half-lives and pharmacokinetic/pharmacodynamic profiles can be critical to therapeutic efficacy. However, because of the diverse chemical nature of biologically active agents, sustained delivery vehicles often need to be specifically designed to accommodate the agent to be delivered and in a manner that is agnostic to the chemical nature of the agent.

Particulate and vesicular biodegradable polymer platforms are an example of promising technologies for the optimisation of prophylactic and therapeutic approaches to a wide variety of diseases and conditions, in particular for immunotherapeutics. Illustrative examples include liposomes, which can be modified to encapsulate small hydrophilic molecules and proteins. However, the stability of these formulations and the release profiles of liposome encapsulated agents cannot be easily controlled. Biodegradable solid particles, on the other hand, are relatively stable and have controllable release characteristics. However, they pose complications for facile encapsulation and controlled release of therapeutic agents. Biodegradable solid particles can also be difficult to administer by injection owing to their relatively high viscosity.

Therefore, despite recent advances, there remains an urgent need for better compositions that can provide a rapid and sustained release profile for biologically active agents.

### SUMMARY OF THE INVENTION

The present disclosure is predicated, at least in part, on the inventors' surprising finding that short biocompatible polymer fibres (SPF) are a suitable biocompatible delivery vehicle for rapid and sustained delivery of biologically active agents. The present inventors have also unexpectedly found that the SPF disclosed herein can protect biologically active agents over an extended period of time and therefore facilitate the rapid and sustained delivery of the biologically active agents over time without compromising the integrity of the biologically active agent.

Thus, in an aspect disclosed herein, there is provided a composition for rapid and sustained delivery of one or more biologically active agents, the composition comprising:

short biocompatible polymer fibres (SPF) having an average length in the range of from about 1 μm to about 3 mm, and an average diameter in the range of from about 15 nm to about 5 μm, wherein the SPF are loaded with one or more biologically active agents,

wherein, when administered, the composition provides sustained release of the one or more biologically active agents from the SPF.

In another aspect disclosed herein, there is provided a method for rapid and sustained delivery of one or more biologically active agents to a subject in need thereof, the method comprising administering to a subject the composition as described above.

In another aspect, there is provided a method for treating or preventing a disease or disorder in a subject in need thereof, the method comprising administering to a subject the composition as described above.

In an embodiment, the composition is administered to the subject subcutaneously.

In another aspect, there is provided a composition as described herein for use in the delivery of the one or more biologically active agents to a subject in need thereof.

In another aspect, there is provided a composition as described herein for use in the treatment or prevention of a disease or disorder when administered to a subject in need thereof.

The present disclosure also extends to use of the composition as described herein in the manufacture of a medicament for the treatment or prevention of a disease or disorder in a subject in need thereof.

In another aspect disclosed herein, there is provided a process for the preparation of a composition for the rapid and sustained delivery of one or more biologically active agents, the process comprising:

(a) introducing a stream of biocompatible polymer fibre-forming liquid into a dispersion medium having a viscosity in the range of from about 1 to 100 centiPoise (cP);
(b) forming a filament in the dispersion medium from the stream of the fibre-forming liquid of (a);
(c) shearing the filament of (b) under conditions allowing fragmentation of the filament and formation of short biocompatible polymer fibres (SPF), wherein the SPF have an average length in the range of from about 1 μm to about 3 mm, and an average diameter in the range of from about 15 nm to about 5 μm; and
(d) loading the SPF of (c) with one or more biologically active agents.

In yet another aspect disclosed herein, there is provided a process for the preparation of a composition for the sustained release of one or more biologically active agents, the process comprising:

(a) providing a mixture comprising (i) a biodegradable polymer fibre-forming liquid and (ii) one or more biologically active agents;
(b) introducing a stream of the mixture of (a) into a dispersion medium having a viscosity in the range of from about 1 to 100 centiPoise (cP);
(b) forming a filament in the dispersion medium from the stream of (a);
(c) shearing the filament of (b) under conditions allowing fragmentation of the filament and formation of short biocompatible polymer fibres (SPF), wherein the SPF have an average length in the range of from about 1 μm to about 3 mm, and an average diameter in the range of from about 15 nm to about 5 μm.

Also disclosed herein is a composition prepared by the process described herein.

The present disclosure also extends to a vaccine composition comprising short biocompatible polymer fibres (SPF), wherein the SPF comprise poly(D,L-lactide-co-glycolide) (PLGA), an average diameter in the range of from about 15 nm to about 5 μm and an average length in the range of from about 1 μm to about 3 mm; and wherein the SPF are loaded with (i) an immunogen selected from the group consisting on a tumour cell lysate and a cancer-associated antigen; (ii) a cytokine and (iii) an adjuvant.

In another aspect, there is provided a vaccine composition comprising short biocompatible polymer fibres (SPF), wherein the SPF comprise poly(D,L-lactide-co-glycolide) (PLGA), an average diameter in the range of from about 15 nm to about 5 μm and an average length in the range of from about 1 μm to about 3 mm; and wherein the SPF are loaded with (i) a tumour cell lysate and/or a cancer-associated antigen of a glioblastoma; (ii) granulocyte-macrophage colony-stimulating factor (GM-CSF); and (iii) a CpG oligonucleotide (CpG).

Further aspects and illustrative embodiments of the invention are also described in the detailed description below.

### BRIEF DESCRIPTION OF THE FIGURES

Illustrative embodiments of the invention will now be described with reference to the following non-limiting figures in which:

FIG. 1 shows photomicrographs showing the incorporation of biological material into the same SPF. Fluorescence images of functionalised SPF containing fluorescently labelled (A) peptide (green/light), (B) 14 kDa protein (blue/light), and (C) DNA (red/light) compared to (D) corresponding bright field images. (E-H) are control unlabelled SPF photographed under the same conditions.

FIG. 2 shows HRP enzyme activity following release from SPF. HRP-SPF were incubated in saline for 7 days. Aliquots were taken at days 1, 2, 3, 6 and 7 and HRP activity was monitored using a colour metric assay, with the resultant colour change measured at 420 nm.

FIG. 3 shows photomicrographs showing the incorporation of OVA into SPF using an OVA antibody and a fluorescent 488 secondary antibody. (A) SPF loaded with OVA protein (fluorescent image—left panel; bright field—right panel); (B) Control/unloaded SPF (fluorescent image—left panel; bright field—right panel).

FIG. 4 shows Biotin-CpG detection within PLGA SPF using an immunoassay. Approximately 100 μg of Biotin-CpGODN was incorporated into 2 ml PLGA to give rise to the SPF. SPF corresponding to 1.5 ml PLGA and 0.5 ml PLGA were added to one well each. SPF were exposed to a HRP-Streptavidin complex. Quantitation was determined using a TMB substrate reagent (3,3′,5,5′-Tetramethylbenzidine) and the resultant colour change was read at 450 nm. The colour change detected indicate biotin-CpGODN was present in the SPF when compared to plain (naked) SPF.

FIG. 5 shows the release kinetics of GM-CSF (pg/mL; y-axis) from SPF over a 28 day period (time/days: x-axis). GMCSF-SPF accumulative release profile is down in (ii). Active GMCSF released by GMSCF-SPF was detected by immuno-assay over a 28 day period. Release profile showed GMCSF release spanned all 28 days with maximum release at 7 days. Approximately 5 μg of GMCSF was added during manufacture of fibres. Standard error bars are shown.

FIG. 6 shows the (i) profile of OVA release from PLGA SPF over a 384 hour time period. Detection was determined by BCA assay. SPF was made with a total of 1.8 mg OVA; (ii) protein release profile over 2 hours. SFP containing 0.5 mg GMSCF or OVA were left to incubate for 2 hours in 1 ml PBS and protein collected. Samples (30 μl, 3 μl and 0.3 μl) were analysed against plain SPF and OVA as standards.

FIG. 7 shows the toxicity of SPF to TF-1 (A) and AML-193 (B) cell lines in the presence of soluble PLGA or SPF over 3 days, compared to 0.5% DMSO, 0.5% PBS or cells alone. Cell viability is shown on the y-axis (cell number). All treatments were non-toxic in culture.

FIG. 8 shows photomicrographs of TF-1 cells (top panels) and AML-193 cells (bottom panels) cultured in the presence (panels A, C, E, G) or absence (B, D, F, H) of SPF. In the presence of SPF, cells exhibited healthy morphology with no observable cell death or disruption to cellular spatial organisation (e.g., did not repel cells).

FIG. 9 shows the biological activity of SPF loaded with GM-CSF. GM-CSF-loaded SPF were incubated whole or dissolved with GM-CSF-starved TF-1 cells (A) or AML-193 cells (B) for 4 days and cell number as measured using MTS assay (abcam, USA). Controls: 5 and 10,000 ng/mL GM-CSF and 0.5% DMSO (carrier for dissolved SPF).

FIG. 10 shows photomicrographs representative of the visualisation of biological activity of GM-CSF-loaded SPF cultured with TF-1 or AML-193 cells for 5 days. Bright field images are shown of TF-1 cells (A) and AML-193 cells (B) grown in the presence of GM-CSF-loaded SPF or unloaded SPF either whole or dissolved in DMSO (×100 magnification).

FIG. 11 shows that SPF protects GM-CSF activity in culture. Cell proliferation rates of AML-193 cells cultured in the presence of GM-CSF (5 mg/mL), GM-CSF added only at the beginning of culture (1000 ng/mL), GM-CSF added fresh every day (5 ng/mL), GM-CSF-loaded SPF and GM-CSF-loaded SPF dissolved in DMSO. the data are compared to controls where cells were cultured in the absence of GM-CSF, in the presence of plain (unloaded SPF), plain (unloaded) SPF dissolved in DMSO and in DMSO alone). Cell numbers are shown on the y-axis; time (days) is shown on the x-axis.

FIG. 12 shows the GM-CSF dose requirement for AML-193 cells in culture. AML-193 cells were GM-CSF starved for 24 hours and subsequently cultured in the absence of GM-CSF or in the presence of GM-CSF at varying concentrations (0.1-10 ng/mL; x-axis). Cell proliferation was measured as an increase in cell number (y-axis) compared to untreated cells after four days in culture. Cell number was determined by MTS assay. The data show that AML-193 cells required greater than 0.5 ng/mL GM-CSF the cell growth. Standard error bars shown.

FIG. 13 shows that SPF protects GM-CSF activity in culture. (A) GM-CSF-loaded SPF were added to cell culture media and subsequently collected and replaced on days 3, 7, 14, 21 and 28 to produce conditioned media. Conditioned media (CM) was then added to GM-CSF-starved TF-1 cells and cell proliferation was measured after 5 days using an MTS assay. Panel (B) shows the GM-CSF dose response of TF-1 cells.

FIG. 14 shows photomicrographs showing that SPF delivery of GM-CSF facilitates dendritic cell differentiation of THP-1 cells. GM-CSF-loaded SPF or plain (unloaded) SPF were incubated in cell culture media for 2 days and then added to the THP-1 monocytic to observe differentiation towards dendritic cells. Differentiation was observed by morphological changes from round (monocytic, white arrows) to elongated cells (dendritic cells; black arrows); (A) GM-CSF positive control; (B) untreated cells); (C) GM-CSF-loaded SPF; (D) plain (unloaded) SPF.

FIG. 15 shows photomicrographs showing that SPF delivery of GM-CSF and CpG drives dendritic cell differentiation of THP-1 monocytic cells. GM-CSF-loaded SPF or SPF loaded with GM-CSF and CpG were incubated in cell culture media for two days and then added to THP-1 cells to observe the extent of differentiation into dendritic cells. Differentiation was observed by morphological changes from round (monocytic, white arrows) to elongated cells (dendritic cells; black arrows); (A) plain (unloaded) SPF; (B) GM-CSF+CpG; (C) GM-CSF-loaded SPF; (D) GM-CSF+CpG-loaded SPF.

FIG. 16 shows the validation of dendritic cell differentiation. Dendritic cell markers CD14 and CD40 were used to monitor differentiation of human monocytes. CD14 expressing monocytes show a decreased expression of CD14 following differentiation, while CD40 expression was increased, indicative of differentiation towards a dendritic cell phenotype.

FIG. 17 shows flow cytometry analysis of cells expressing both CD8+ and SIINFEKL T-cell surface recognition markers. (A-H) Vaccine SPF, (I-0) plain SFP, (P-S) vaccine alone, (T-V) saline.

FIG. 18 shows detection of OVA T cells. Vaccine administered using SPF shows higher OVA activated T cells compared to SFP (P<0.01) or saline alone (P<0.01). Unloaded SFP behaved the same way as saline controls. Bars equal range. (n=8 vaccine SFP; n=7 plain SPF; n=4 vaccine alone; n=3 saline).

FIG. 19 shows flow cytometry analysis CD8+ T cells expressing IFNγ when challenged with SIIKFEKL peptide. (A-H) Plain SPF, (I-P) vaccine SFP, (Q-R) saline alone.

FIG. 20 shows detection of cytotoxic T cells. IFNγ was detected in cells challenged with SIIFEKL peptide identifying a higher number of cytotoxic cells in the mice administered vaccine via SFP compared to SFP alone (P<0.0001), saline alone (P<0.005) or vaccine alone (P<0.05). Saline control showed the absence of cytotoxic cells. Bars equal range. (n=8 SFP and SPF vaccine; n=4 vaccine only; n=2 saline). Note one saline mouse was determined to have an unrelated infection.

FIG. 21 shows EliSpot assay of spleenoctyes expressing IFNγ when challenged with the SINFEKL peptide. Mouse 1-4 received injection of SFP+OVA (Drug), while mouse 5 and 6 received SPF only.

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described.

The articles “a” and “an” are used herein to refer to one or to more than one (i.e., to at least one) of the grammatical object of the article. By way of example, “an immunogen” means one immunogen or more than one immunogen; “a cytokine” means one cytokine or more than one cytokine; and so on.

As used herein, the term “about” refers to a quantity, level, value, dimension, size, or amount that varies by as much as 10% (e.g, by 10%, 9%, 8%, 7%, 6%, 5%, 4%, 3%, 2% or 1%) to a reference quantity, level, value, dimension, size, or amount.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

As disclosed elsewhere herein, the present disclosure is predicated, at least in part, on the inventors' surprising finding that short biocompatible polymer fibres (SPF) are a suitable biocompatible delivery vehicle for the rapid and sustained delivery of biologically active agents. The present inventors have also unexpectedly found that compositions of SPF can protect the biologically active agents over an extended period of time and are therefore able to facilitate the rapid and sustained delivery of active agents without compromising the integrity of the active agent.

Thus, in an aspect disclosed herein, there is provided a composition for rapid and sustained delivery of one or more biologically active agents, the composition comprising:

short biocompatible polymer fibres (SPF) having an average length in the range of from about 1 μm to about 3 mm, and an average diameter in the range of from about 15 nm to about 5 μm, wherein the SPF are loaded with one or more biologically active agents,

wherein, when administered, the composition provides sustained release of the one or more biologically active agents from the SPF.

### Short Biocompatible Polymer Fibres

The terms “short biocompatible polymer fibres”, “short polymer fibres” and “SPF” are used interchangeably herein to describe short polymeric fibres having an average length in the range of from about 1 μm to about 3 mm, and an average diameter in the range of from about 15 nm to about 5 μm.

In an embodiment, the SPF have an average diameter in the range of from about 40 nm to about 5 μm, or preferably from about 50 nm to about 3 μm. In an embodiment, the SPF have an average diameter in the range of from about 100 nm to about 2 μm. In a preferred embodiment, the SPF have an average diameter in the range of from about 15 nm to about 5 μm. In another embodiment, the SPF have an average diameter in the range of from about 50 nm to about 500 nm. In a more preferred embodiment, the SPF have an average diameter in the range of from about 50 nm to about 300 nm.

The average diameter of the SPF may be influenced by parameters such as shear stress, the quantity of the fibre-forming substance and the temperature(s) during manufacture. Accordingly, these parameters can be varied to obtain SPF of the desired average diameter or range of diameters. For example, a lower polymer concentration will typically provide SPF having a smaller average diameter, all other parameters being equal. The polydispersity of the SPF can be reduced by optimizing the experimental parameters described above. Hence, the average diameter of the SPF will typically be determined by the parameters set during manufacture, as is described, for example, in WO 2013/056312, and will have a controllable diameter as substantially determined by such factors.

In an embodiment, the SPF will have a monodispersed diameter, whereby each of the SPF will have the same, or substantially the same, diameter. It will be understood, however, that the SPF of the compositions described herein do not need to have the same, or substantially the same, diameter, and that the SPF will likely function in a similar way to provide for rapid and sustained delivery of the one or more biological agents loaded therein as long as they have an average fibre diameter within the ranges described herein.

In an embodiment, the SPF of the compositions described herein will have a bimodal or multimodal fibre diameter distribution. This may be achieved by varying the injection speed or shear rate during injection of the fibre-forming liquid in the dispersant, as is described, for example, in WO 2013/056312. The SPF of the compositions described herein may have a low distribution of fibre diameters (i.e., a narrow polydispersity). In an embodiment, the SPF comprise a distribution of diameters that deviates by no more than about 50%, preferably by no more than about 45%, even more preferably by no more than about 40%, from the average diameter of the SPF of the composition.

SPF of the compositions disclosed herein may be formed of any length, and a wide distribution of lengths can also be obtained. In an embodiment, the SPF of the compositions described herein have an average length of at least about 1 μm. In an embodiment, the SPF of the compositions described herein have an average length in the range of from about 1 μm to about 3 mm. In an embodiment, the SPF have an average length in the range of from about 1 μm to about 20 μm. In a preferred embodiment, the SPF have an average length in the range of from about 1 μm to about 10 μm. The shear stress applied to the filament may affect the length of the resulting SPF, with high shear stress typically providing shorter fibre lengths. Fibre lengths of the SPF may therefore be adjusted by varying the operating parameters, as described herein.

In some embodiments, the SPF will have a monodispersed length, whereby each of the SPF will have the same, or substantially the same, length. It will be understood that the SPF of the composition described herein do not need to have the same, or substantially the same, length, and that the SPF will likely function in a similar way to provide for the rapid and sustained delivery of the one or more biological agents dispersed or loaded therein as long as they have fibre lengths typically in the range of from about 1 μm to about 3 mm.

In an embodiment, the SPF will have a bimodal or multimodal fibre length distribution. This may be achieved by varying the injection speed or shear rate during injection of the fibre-forming liquid in the dispersant, as is described, for example, in WO 2013/056312. The SPF of the compositions described herein may have a low distribution of fibre lengths (i.e., a narrow polydispersity). In an embodiment, the SPF comprise a distribution of fibre lengths that deviates by no more than about 50%, preferably by no more than about 45/a, even more preferably by no more than about 40%, from the average diameter of the SPF of the composition.

Illustrative examples of suitable SPF are described in WO 2013/056312, the contents of which are incorporated herein by reference in their entirety. In an embodiment, the SPF have the fibre diameter and length characteristics of the SPF that are described in WO 2013/056312, or produced by the methods described in WO 2013/056312.

The SPF will have a substantially elongated shape, typically a substantially cylindrical shape. The physical characteristics of the SPF, such as shape, diameter and length, can be determined using conventional techniques known to persons skilled in the art, illustrative examples of which include optical microscopy or scanning electron microscopy.

The SPF may suitably be crosslinked. To form crosslinked SPF, crosslinking agents may be included in a fibre-forming solution and/or in the dispersion medium during the SPF manufacturing process. Illustrative examples of suitable crosslinking agents that may be used include glutaraldehyde, paraformaldehyde, homo-bifunctional or hetero-bifunctional organic crosslinkers, and multi-valent ions such as Ca²⁺, Zn²⁺, Cu²⁺. The selection of crosslinking agent may depend on the nature of the fibre-forming substance used to the form the SPF. Crosslinking of the as-formed SPF resident in the dispersion medium may occur by suitable initiation of the crosslinking reaction, for example, by addition of an initiator molecule or by exposure to an appropriate wavelength of radiation, such as UV light. Crosslinking of the SFP can be useful to further improve the stability of the SPF such that they can be readily transferred from one medium to another if desired.

The terms “sustained”, “sustained release” and “sustained delivery” are used interchangeably herein to mean the delivery of the one or more biologically active agents subsequent to administration or delivery, typically in vivo, whereby the rate of release of the agent(s) from the SPF is slower than would otherwise occur if the agent(s) was/were administered to the subject directly (i.e., in the absence of the SPF). Sustained release will typically occur over a time period that is substantially longer than for rapid delivery. The sustained release of the one or more biologically active agents, as described herein, will typically provide a dose of the one or more biologically active agents over a longer period of time and therefore aid in prolonging the biological (e.g., therapeutic) effect provided by the one or more biologically active agents. In some embodiments, sustained release of the one or more biologically active agents occurs over a period of at least 24 hours (e.g., 24 hrs, 2 days, 3 days, 4 days, 5 days, 6 days, 7 days, 8 days, 9 days, 10 days, 11 days and so on). In an embodiment, the composition provides sustained release of the one or more biologically active agents over a period of at least 24 hours, preferably over a period of at least 3 days, preferably over a period of at least 7 days, preferably over a period of at least 14 days, preferably over a period of at least 21 hours, or more preferably over a period of at least 28 days following administration. In an embodiment, the composition provides a peak release of the one or more biologically active agents over a period from about 3 days to about 14 days following administration, preferably over a period from about 4 days to about 9 days, more preferably at about 7 days following administration. In some embodiments, sustained release of the one or more biologically active agents occurs over a period of at least one day, preferably over at least one week, or more preferably over at least one month.

The present inventors have also unexpectedly shown that the SPF disclosed herein have advantageous properties that make them suitable as a sustained delivery vehicle for biological agents, including that they (i) have sufficiently low viscosity in solution to allow for administration by injection and (ii) are non-toxic (or substantially non-toxic) to cells, including immune cells.

In some embodiments, the SPF can be suitably made from “smart” polymers, such as temperature- or pH-responsive polymer material or biopolymers (e.g., collagen, chitosan, gelatin, or mixtures of these) to give additional unique properties that can be manipulated for a desired application. Suitable smart polymers, including temperature- and pH-responsive polymer material, will be familiar to persons skilled in the art, illustrative examples of which are described in Cohen Stuart et al. (2010; Nature Materials, 9:101-113), the contents of which are incorporated herein by reference in their entirety.

The SPF can also be functionalized using, for example, standard wet chemistry (e.g., by binding functional groups to the surface of the fibres), so as to allow for the attachment of active moieties, including the biologically active agents as described elsewhere herein. Suitable methods for functionalising polymer material that can be applied to the SPF disclosed herein will be familiar to persons skilled in the art, illustrative examples of which are described in Gong and Chen (2016; Saudi Pharm. J. 24(3): 254-257).

The SPF described herein can be used as carriers of a variety of biologically active agents, as is described elsewhere herein. These can range from functional small molecules (i.e., drugs, herbicides, etc.) to larger biomolecules (e.g., proteins, peptides, enzymes, oligos, etc.). The active agents can be loaded onto the polymer fibres after the production of the SPF. Alternatively, or in addition, the active agents can be loaded into the polymer fibres during the production of the SPF, as is described, for example, in WO 2013/056312.

The term “loaded” is to be understood to mean that the one or more biologically active agents are integrated, incorporated, dispersed or otherwise in close associated with the SPF, whereby the active agents are released from the SPF upon delivery of the compositions, e.g., in vivo. Without being bound by theory or by a particular mode of action, the sustained release of the biologically active from the SPF is attributed, at least in part, to the degradation of the SPF over time, in particular when the loaded SPF are exposed to an environment that promotes the degradation of SPF over time as would be the case where the SPF are administered to a subject subcutaneously, intramuscularly, transdermally (e.g., via a transdermal patch) or intravascularly. The sustained release of the biologically active from the SPF may also be attributed, at least in part, to the diffusion of the active agents into the environment from the SPF in a manner that is independent of the degradation of the SPF. In an embodiment disclosed herein, the composition is an injectable composition. In an embodiment, the composition is formulated for administration through a 22-25 gauge needle.

### Biocompatible Polymers

The SPF may be formed from any suitable biocompatible polymer, illustrative examples of which will be known to persons skilled in the art.

As used herein, the term “biocompatible polymer” typically refers to a polymer material that, when introduced into a biological system (e.g., in vitro, ex vivo or in vivo), will have no, or substantially no, adverse impact on the biological system or on a part thereof. By “substantially no adverse impact” is to be understood to mean that the polymer may have some (negative and/or positive) impact on the biological system to which it comes into contact, but the extent of any such impact will be minimal and will not result, for example, in a reduction in the therapeutic efficacy of the composition.

The biocompatible polymer can be a synthetic or a natural (i.e., naturally-occurring) polymer. Illustrative examples of suitable natural polymers include proteins such as albumin, collagen, gelatin and prolamines, for example, zein, and polysaccharides such as alginate, cellulose derivatives and polyhydroxyalkanoates, for example, polyhydroxybutyrate.

The biocompatible polymer may be a biodegradable polymer, a non-biodegradable polymer, or substantially non-biodegradable polymer. It would be understood, however, that it is generally desirable that the biocompatible polymer is biodegradable, or substantially biodegradable, so as to avoid or minimise the impact the polymer may otherwise have on a biological system over time.

In an embodiment, the biocompatible polymer is a biodegradable polymer. Suitable biodegradable polymers will be known to persons skilled in the art, illustrative examples of which polypeptides, alginates, chitosan, starch, collagen, silk fibroin, polyurethanes, polyacrylic acid, polyacrylates, polyacrylamides, polyesters, polyolefins, boronic acid functionalised polymers, polyvinylalcohol, polyallylamine, polyethyleneimine and polyvinyl pyrrolidone), poly(lactic acid), polyether sulfone, inorganic polymers, and a combination of any of foregoing. Thus, in an embodiment disclosed herein, the biodegradable polymer is selected from the group consisting of polypeptides, alginates, chitosan, starch, collagen, silk fibroin, polyurethanes, polyacrylic acid, polyacrylates, polyacrylamides, polyesters, polyolefins, boronic acid functionalised polymers, polyvinylalcohol, polyallylamine, polyethyleneimine and polyvinyl pyrrolidone), poly(lactic acid), polyether sulfone, inorganic polymers, and a combination of any of foregoing.

The biodegradable polymer can be selected to degrade over a time period ranging from one day to more than one year, more preferably from seven days to 26 weeks, more preferably from seven days to 20 weeks, or most preferably from seven days to 16 weeks. It will be understood that the choice of polymer may depend on the intended use. In some embodiments, a synthetic polymer may be preferred. In other embodiments, a natural polymer may be preferred. Other illustrative examples of suitable polymers include poly(lactic acid), poly(glycolic acid), poly(lactic acid-co-glycolic acids), polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); poly(glycolide-co-caprolactones); polycarbonates such as tyrosine polycarbonates; polyamides (including synthetic and natural polyamides), polypeptides, and poly(amino acids); polyesteramides; other biocompatible polyesters; poly(dioxanones); poly(alkylene alkylates); hydrophilic polyethers; polyurethanes; polyetheresters; polyacetals; polycyanoacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), polyvinyl alcohols, polyvinylpyrrolidone; poly(alkylene oxides) such as polyethylene glycol (PEG); derivativized celluloses such as alkyl celluloses (e.g., methyl cellulose), hydroxyalkyl celluloses (e.g., hydroxypropyl cellulose), cellulose ethers, cellulose esters, nitrocelluloses, polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), as well as derivatives, copolymers, and blends thereof. As used herein, “derivatives” include polymers having substitutions, additions of chemical groups and other modifications to the polymeric backbones described above routinely made by those skilled in the art. Natural polymers, including proteins such as albumin, collagen, gelatin, prolamines, such as zein, and polysaccharides such as alginate and pectin, may also be incorporated into the SPF.

In an embodiment, the biocompatible polymer is selected from the group consisting of polypeptides, alginates, chitosan, starch, collagen, silk fibroin, polyurethanes, polyacrylic acid, polyacrylates, polyacrylamides, polyesters, polyolefins, boronic acid functionalised polymers, polyvinylalcohol, polyallylamine, polyethyleneimine and polyvinyl pyrrolidone), poly(lactic acid), polyether sulfone, inorganic polymers, and a combination of any of foregoing.

In an embodiment, the biocompatible polymer comprises poly(lactic acid).

In another embodiment, the poly(lactic acid) is poly(lactic-co-glycolic acid) (PLGA). In some embodiments, the poly(lactic-co-glycolic acid) is poly(D,L-lactide-co-glycolide).

In an embodiment, the poly(lactic-co-glycolic acid) has a lactide:glycolide ratio of about 85:15.

In an embodiment, the poly(lactic-co-glycolic acid), for example poly(D,L-lactide-co-glycolide), has an Mw from about 50 kDa to about 75 kDa. In another embodiment, the poly(lactic-co-glycolic acid) has an Mw from about 190 kDa to about 240 kDa. In one embodiment, the biocompatible polymer comprises a combination of a first poly(lactic-co-glycolic acid) polymer component having an Mw from about 50 kDa to about 75 kDa and a second poly(lactic-co-glycolic acid) polymer component having an Mw from about 190 kDa to about 240 kDa. Mw is the weight average molecular weight of the polymer. In an embodiment, the PLGA may suitably comprise a combination of different forms of PLGA, including those described herein. Preferably, the different forms of PLGA may be combined in proportions or absolute amounts suitable to produce the SPF with the desired properties as herein described. Suitable combinations can be ascertained using methods known to persons skilled in the art, illustrative examples of which include combinations of poly(D,L-lactide-co-glycolide) having an Mw from about 50 kDa to about 75 kDa and poly(D,L-lactide-co-glycolide) having an Mw from about 190 kDa to about 240 kDa. Thus, in an embodiment, the PLGA comprises poly(D,L-lactide-co-glycolide) having an Mw from about 50 kDa to about 75 kDa and poly(D,L-lactide-co-glycolide) having an Mw from about 190 kDa to about 240 kDa. In another embodiment, the PLGA comprises from about 5% to about 50% poly(D,L-lactide-co-glycolide) having an Mw from about 50 kDa to about 75 kDa and from about 50% to about 95% poly(D,L-lactide-co-glycolide) having an Mw from about 190 kDa to about 240 kDa. In another embodiment, the PLGA comprises from about 5% to about 20% poly(D,L-lactide-co-glycolide) having an Mw from about 50 kDa to about 75 kDa and from about 80% to about 95% poly(D,L-lactide-co-glycolide) having an Mw from about 190 kDa to about 240 kDa. In yet another embodiment, the PLGA comprises from about 10% poly(D,L-lactide-co-glycolide) having an Mw from about 50 kDa to about 75 kDa and about 90% poly(D,L-lactide-co-glycolide) having an Mw from about 190 kDa to about 240 kDa.

In an embodiment, the composition comprises one or more crosslinkable SPF comprising one or more photo-polymerizable groups, allowing for the crosslinking of the SPF in suspension. Illustrative examples of suitable photo-polymerizable groups include vinyl groups, acrylate groups, methacrylate groups, and acrylamide groups. Photo-polymerizable groups, when present, may be incorporated within the backbone of the crosslinkable SPF, within one or more of the sidechains of the crosslinkable SPF, at one or more of the ends of the crosslinkable SPF, or combinations thereof.

In an embodiment, the SPF comprises 1% w/v Resomer® RG 858 S (an ester-terminated Poly(D,L-lactide-co-glycolide, lactide:glycolide 85:15, Mw 190-240 kDa) and 0.234% Poly(D,L-lactide-co-glycolide). The 0.234% Poly(D,L-lactide-co-glycolide may have a Mw of 50-75 kDa.

The SPF may suitably comprise at least one additive. The additive may be introduced to the SPF by incorporating at least one additive in the polymer fibre-forming liquid and/or the dispersion medium used to prepare the SPF. The additive may be included during the manufacturing/extrusion process in the fibre-forming liquid and/or dispersion medium. Alternatively, or in addition, the additive may be introduced to the SPF by adding it to the SPF subsequent to their manufacture. Illustrative examples of suitable additives include colorants (e.g. fluorescent dyes and pigments), odorants, deodorants, plasticizers, impact modifiers, fillers, nucleating agents, lubricants, surfactants, wetting agents, flame retardants, ultraviolet light stabilizers, antioxidants, biocides, thickening agents, heat stabilizers, defoaming agents, blowing agents, emulsifiers, crosslinking agents, waxes, particulates, flow promoters, coagulating agents (including: water, organic and inorganic acids, organic and inorganic bases, organic and inorganic salts, proteins, coordination complexes and zwitterions), multifunctional linkers (such as homo-multifunctional and hetero-multifunctional linkers) and other materials added to enhance processability or end-use properties of the polymeric components. Such additives can be used in conventional amounts that will be known to persons skilled in the art.

### Biologically Active Agents

As used herein, the term “biologically active agent” refers to any molecule of synthetic or natural origin that is capable of eliciting a physiological response in a biological system, whether in vitro, ex vivo or in vivo.

By “one or more biologically active agent” is meant 1, 2, 3, 4, 5, 6, 7, and so on, biologically active agents. In an embodiment, the composition comprises at least 1 biologically active agent, preferably at least 2 biologically active agents, preferably at least 3 biologically active agents, preferably at least 4 biologically active agents, preferably at least 5 biologically active agents, preferably at least 6 biologically active agents, preferably at least 7 biologically active agents, preferably at least 8 biologically active agents, preferably at least 9 biologically active agents, or more preferably at least 10 biologically active agents.

Suitable biologically active agents will be known to persons skilled in the art, the choice of which will likely depend on the intended therapeutic, prophylactic and/or diagnostic use of the compositions disclosed herein, such as the nature or type of disease or disorder to be treated. Illustrative examples of suitable biologically active agents include small molecule drugs, hormones, antimicrobial compounds, antimicrobial proteins, antivirals, steroids, chemotherapy drugs, ligands, binding agents (e.g., aptamers, small interfering RNA, antibodies and antigen-binding fragments thereof, including therapeutic antibodies and antigen-binding fragments thereof), cell lysates, cytokines, growth factors, fusion proteins, immunogens, antigens, viruses, viral proteins, bacteria, bacterial proteins and fragments thereof, bacteria cell lysates, hormones and nucleic acid molecules, including nucleic acid molecules encoding any one or more of the foregoing. It is to be understood that the compositions disclosed herein may comprise one or more biologically active agents selected from one or more classes, including from one or more of the aforementioned classes.

Conversely, when the compositions disclosed herein comprise two or more biologically active agents, the biologically active agents may belong to the same class of active agents.

In an embodiment, the one or more biologically active agents are selected from the group consisting of a hormone, an antimicrobial agent, an antiviral, a steroid, a chemotherapy drug, a therapeutic binding agent (e.g., an aptamer, an antibody or antigen-binding fragments thereof), a cytokine, an immunogen and a nucleic acid molecule.

In an embodiment, the one or more biologically active agents comprises an immunogen. The term “immunogen” is understood to mean a peptide or protein that is capable of raising an immune response, including a humoral (antibody) response, in vivo. The terms “peptide”, “polypeptide” and “protein” are used interchangeably herein in their broadest sense to refer to a molecule of two or more amino acid residues, or amino acid analogs. The amino acid residues may be linked by peptide bonds, or alternatively by other bonds, e.g. ester, ether etc., but in most cases will be linked by peptide bonds. The terms “amino acid” or “amino acid residue” are used herein to encompass both natural and unnatural or synthetic amino acids, including both the D- or L-forms, and amino acid analogs. An “amino acid analog” is to be understood as a non-naturally occurring amino acid differing from its corresponding naturally occurring amino acid at one or more atoms. For example, an amino acid analog of cysteine may be homocysteine. Suitable immunogens will be familiar to persons skilled in the art, noting that the choice of immunogen will also largely depend on the intended therapeutic or prophylactic use. Illustrative examples of suitable immunogens include a tumour cell, a tumour cell lysate, a virus, a viral antigen, a bacteria, a bacteria cell lysate, a cancer-associated antigen and nucleic acid molecules encoding any one or more of the foregoing. Thus, in an embodiment disclosed herein, the immunogen is selected from the group consisting of a tumour cell, a tumour cell lysate, a virus, a viral antigen, a bacteria, a bacteria cell lysate, a cancer-associated antigen and nucleic acid molecules encoding any one or more of the foregoing.

In another embodiment, the one or more biologically active agents comprises a fusion protein. The term “fusion protein”, as used herein, typically refers to two or more peptide sequences (e.g., immunogens) linked in such a way as to produce a peptide that would not otherwise occur in nature. In an embodiment, the fusion protein comprises two or more peptide sequences linked to one another end-to-end. In an embodiment, the fusion protein comprises two or more peptide sequences linked to one another in a linear configuration via a suitable linking moiety, also referred to herein as a linker. Suitable methods of linking peptide sequences will be familiar to persons skilled in the art, illustrative examples of which include peptide (amide) bonds and linkers. As used herein, the term “linker” refers to a short polypeptide sequence interposed between any two neighboring peptide sequences as herein described. In an embodiment, the linker is a polypeptide linker of 1 to 10 amino acids, preferably 1, 2, 3, 4 or 5 naturally or non-naturally occurring amino acids. In an embodiment, the linker is a carbohydrate linker. Suitable carbohydrate linkers will be known to persons skilled in the art. In another embodiment disclosed herein, the fusion protein comprises one or more peptidic or polypeptidic linker(s) together with one or more other non-peptidic or non-polypeptidic linker(s). Further, different types of linkers, peptidic or non-peptidic, may be incorporated in the same fusion peptide as deemed appropriate. In the event that a peptidic or polypeptidic linker is used to join two respective peptide sequences, the linker will be advantageously incorporated such that its N-terminal end is bound via a peptide bond to the C-terminal end of the one peptide sequence, and its C-terminal end via a peptide bond to the N-terminal end of the other peptide sequence. The individual peptide sequences within the fusion protein may also have one or more amino acids added to either or both ends, preferably to the C-terminal end. Thus, for example, linker or spacer amino acids may be added to the N- or C-terminus of the peptides or both, to link the peptides and to allow for convenient coupling of the peptides to each other and/or to a delivery system such as a carrier molecule serving as an anchor. An illustrative example of a suitable peptidic linker is LP (leucine-proline). Also contemplated herein are fusion proteins comprising at least two of the peptide sequences concatenated two or more times in tandem repeat. Without being bound by theory or by a particular mode of application, it will be understood that incorporating two or more different peptide sequences into the fusion peptide, as herein described, may generate a more beneficial immune response by eliciting a higher antibody titre as compared to an immunogen comprising a single peptide sequence disclosed herein. Suitable methods of preparing a fusion protein, as herein described, would be familiar to persons skilled in the art. An illustrative example includes peptide synthesis that involves the sequential formation of peptide bonds linking each peptide sequence, as herein described, to its respectively neighboring peptide sequence, and recovering said fusion peptide. Illustrative examples include the methods described in “Amino Acid and Peptide Synthesis” (Oxford Chemistry Primers; by John Jones, Oxford University Press).

Synthetic peptides can also be made by liquid-phase synthesis or solid-phase peptide synthesis (SPPS) on different solid supports (e.g. polystyrene, polyamide, or PEG). SPPS may incorporate the use of F-moc (9H-fluoren-9-ylmethoxycarbonyl) or t-Boc (tert-Butoxycarbonyl). Custom peptides are also available from a number of commercial manufacturers. Alternatively, the fusion protein may be prepared by recombinant methodology. For example, a nucleic acid molecule comprising a nucleic acid sequence encoding the fusion protein can be transfecting into a suitable host cell capable of expressing said nucleic acid sequence, incubating said host cell under conditions suitable for the expression of said nucleic acid sequence, and recovering said fusion protein. Suitable methods for preparing a nucleic acid molecule encoding the fusion protein will also be known to persons skilled in the art, based on knowledge of the genetic code, possibly including optimizing codons based on the nature of the host cell (e.g. microorganism) to be used for expressing and/or secreting the recombinant fusion protein. Suitable host cells will also be known to persons skilled in the art, illustrative examples of which include prokaryotic cells (e.g., E. coli) and eukaryotic cells (e.g., P. pastoris). Reference is made to “Short Protocols in Molecular Biology, 5th Edition, 2 Volume Set: A Compendium of Methods from Current Protocols in Molecular Biology” (by Frederick M. Ausubel (author, editor), Roger Brent (editor), Robert E. Kingston (editor), David D. Moore (editor), J. G. Seidman (editor), John A. Smith (editor), Kevin Struhl (editor), J Wiley & Sons, London).

In an embodiment, the immunogen is a tumour cell lysate. Persons skilled in the art will understand that the choice of tumour cell lysate will depend on the type of disease or disorder to be treated or prevented. The tumour cell lysate will typically be prepared from a sample of tumour cell derived from the cancer. For instance, where the cancer is a cancer of the liver, the tumour cell lysate may suitably be prepared from one or more cancer cells derived from the tumour in the subject to be treated. In an embodiment, the tumour cell is a glioblastoma tumour cell. In a preferred embodiment, the glioblastoma is glioblastoma multiforme.

In an embodiment, the one or more biologically active agents comprises a cytokine. Suitable cytokines will be known to persons skilled in the art, illustrative examples of which includes interleukin 4 (IL-4) and granulocyte-macrophage colony-stimulating factor (GM-CSF). Thus, in an embodiment disclosed herein, the cytokine is GM-CSF.

In another embodiment, the one or more biologically active agents comprises a hormone. Suitable hormones will be known to persons skilled in the art, illustrative examples of which include insulin and somatotropin, and steroid hormones such as corticosteroids, estrogens, progestogens and androgens. The present disclosure also extends to the use of peptide hormones. A “peptide hormone” is typically understood to be a peptide or protein that has an effect on the endocrine system of a subject. An illustrative example of a suitable peptide hormone is somatotropin. Somatotropin stimulates the growth, cell reproduction and cell regeneration in humans and non-human animals and is important in growth and development.

In another embodiment disclosed herein, the one or more biologically active agents comprises a binding agent, illustrative examples of which will be known to persons skilled in the art and include aptamers, antibodies, and antigen-binding fragments thereof. The binding agent may be a therapeutic antibody to a target antigen of interest, such as a viral protein or a cancer-associated antigen. In other embodiments, the antibody may be used to target the loaded SPF to a biological site of interest (i.e., a targeting antibody or binding fragment thereof).

In an embodiment, the one or more biologically active agent comprises a cancer-associated antigen. The terms “cancer-associated antigen”, “antigen associated with cancer, “tumour-associated antigen”, “tumour antigen”, “cancer antigen” and the like are used interchangeably herein to mean an antigen that is aberrantly expressed in cancer cells or tissue. In some embodiments, the antigen may be expressed under normal conditions in a limited number of tissues and/or organs or in specific developmental stages. For example, the antigen may be specifically expressed under normal conditions in stomach tissue and is expressed or aberrantly expressed (e.g., overexpressed) in one or more cancer cells. The expression of antigen may be reactivated in cancer cells or tissue irrespective of the origin of the cancer. In some embodiments, the cancer-associated antigen includes differentiation antigens, preferably cell type-specific differentiation antigens (i.e., proteins that are specifically expressed under normal conditions in a certain cell type at a certain differentiation stage), cancer/testis antigens (i.e., proteins that are specifically expressed under normal conditions in testis and sometimes in placenta), and germline specific antigens.

In an embodiment, the cancer-associated antigen is expressed on the cell surface of a cancer cell and is preferably not or only rarely expressed on normal cells and tissues. Preferably, the antigen or the aberrant expression of the antigen identifies cancer cells, preferably tumour cells. In some embodiments, the antigen that is expressed by a cancer cell in a subject (e.g., a patient suffering from cancer) is a self-protein. It will be understood, however, that no autoantibodies directed against the antigen are typically found in a detectable level under normal conditions in a subject carrying the antigen (typically a healthy patient that does not have cancer) or such autoantibodies can only be found in an amount below a threshold concentration that would be necessary to damage the tissue or cells carrying the antigen. Suitable cancer-associated antigens will be known to persons skilled in the art, illustrative examples of which include EGFR (e.g., Her2/neu, Her-1), BAGE (B melanoma antigen), CEA (carcinoembryonic antigen), Cpg (cytosine-phosphate diesterguanine), Gp100 (glycoprotein 100), h-TERT (telomerase transcriptase), MAGE (melanoma antigen-encoding gene), Melan-A (melanoma antigen recognized by T cells) and MUC-1 (mucin-1). Thus, in an embodiment, the cancer-associated antigen is selected from the group consisting of EGFR (e.g., Her2/neu, Her-1), BAGE (B melanoma antigen), CEA (carcinoembryonic antigen), CpG (cytosine-phosphate diesterguanine), Gp100 (glycoprotein 100), h-TERT (telomerase transcriptase), MAGE (melanoma antigen-encoding gene), Melan-A (melanoma antigen recognized by T cells) and MUC-1 (mucin-1). It will also be understood that the choice of antigen that is to be the target of the vaccine composition produced by the methods disclosed herein will typically depend on the intended use of the vaccine composition. For example, if the vaccine composition is intended to treat subjects with breast cancer, then the antigen will typically be an antigen that is associated with (e.g., overexpressed by) the breast cancer. Suitable examples of antigens associated with breast cancer will be familiar to persons skilled in the art, illustrative examples of which include the epidermal growth factor receptors Her2/neu and Her1. Other illustrative examples of suitable cancer-associated antigens include Wilms tumor-1 (WT1), survivin and cytomegalovirus (CMV).

In an embodiment disclosed herein, the one or more biologically active agents comprises a cancer-associated antigen selected from the group consisting of Wilms tumor-1 (WT1), survivin and cytomegalovirus (CMV).

The amount of the one or biologically active agents in the loaded SPF of the compositions described herein will vary, depending on, for example, the solubility of the SPF and the characteristics of the biologically active agent(s) (e.g., size, net charge, molecular weight of the biologically active agent(s)). This is also referred to herein as the “loading rate”; that is, the amount of the biologically active agent(s) in the loaded SPF as a proportion of the total weight of the loaded SPF. In an embodiment, the amount of the one or more biologically active agents in the loaded SPF is from about 5% to about 95% by weight of the total weight of the loaded SPF. In an embodiment, the amount of the one or more biologically active agents in the loaded SPF is from about 10/to about 60% by weight of the total weight of the loaded SPF. In an embodiment, the amount of the one or more biologically active agents in the loaded SPF is from about 20% to about 50% by weight of the total weight of the loaded SPF. In an embodiment, the amount of the one or more biologically active agents in the loaded SPF is from about 30% to about 50% by weight of the total weight of the loaded SPF.

In an embodiment disclosed herein, the one or more biologically active agents are incorporated into the SPF indirectly by attachment to a linker or other functional moiety incorporated into the SPF. Suitable linkers and functional moieties will be familiar to persons skilled in the art, illustrative examples of which include biotin, streptavidin, immunoglobulins and antigen-binding fragments thereof (e.g., Fab, scFv) and nucleic acid molecules. For example, the SPF may be loaded with biotin and the biotin-loaded SPF subsequently combined with one or more biologically active agents to which streptavidin has been attached, whereby the streptavidin-agent complex binds to the biotin within the loaded SPF to produce SPF loaded with the one or more biologically active agents.

Similarly, the SPF may be loaded with an immunoglobulin, or an antigen-binding fragment thereof, that specifically binds a biologically active agent and the loaded SPF subsequently combined with the biologically active agent under conditions to allow the agent to bind to the immunoglobulin or antigen-binding fragment thereof to produce SPF loaded with the one or more biologically active agents. The present disclosure extends to embodiments where the linker or functional moiety binds to the one or more biologically active agents via covalent or non-covalent forces.

### Adjuvants

In an embodiment, the one or more biologically active agents comprise an adjuvant. The term “adjuvant”, as used herein, refers to a compound or substance that is capable of enhancing a subject's physiological response to the one or more biologically active agents. Where the one or more biologically active agents comprises an immunogen, an adjuvant may act to enhance a subject's immune response to the immunogen by increasing the antibody response to the immunogen and thus the longevity of the immune response. An adjuvant can therefore help to promote a more effective physiological response to the one or more biologically active agents in a subject, compared to the administration of the one or more biologically active agents alone or in the absence of the adjuvant.

In some embodiments, an adjuvant may act to modify the release of a biologically active agent in vivo. The modulated release can provide a more durable or higher level of delivery using smaller amounts or fewer doses of the biologically active agent, compared to if the biologically active agent were administered alone or without the adjuvant.

The adjuvant can be present in the water-in-oil emulsion of the composition and may be in the oil phase or in the aqueous phase of the emulsion. In some embodiments of the composition, the oil phase and aqueous phase of the emulsion may each comprise an adjuvant.

In one embodiment disclosed herein, the adjuvant is hydrophilic and water soluble. Suitable hydrophilic adjuvants will be familiar to persons skilled in the art, illustrative examples of which include alum, the water soluble extract of Mycobacterium smegmatis, synthetic N-acetyl-muramyl-l-alanyl-d-isoglutamine, monoacyl lipopeptides and ligands for Toll-like receptors. Such adjuvants may be incorporated in the aqueous liquid and/or within hydrogel particles of the aqueous phase.

In an embodiment, the adjuvant is lipophilic and oil soluble. In some embodiments, the oil per se can be an adjuvant and thus the oil phase comprises an adjuvanting oil. The use of an adjuvanting oil may be desirable as it avoids the need to incorporate a separate adjuvanting compound in the composition of the invention. Illustrative examples of suitable adjuvanting oils will be familiar to persons skilled in the art.

In other embodiments, the adjuvant is a lipophilic adjuvant dissolved or suspended in a non-adjuvanting (passive) oil.

As noted elsewhere herein, suitable adjuvants are known to those skilled in the art. Adjuvants useful for the composition disclosed herein may be inorganic adjuvants or organic adjuvants. A skilled person would appreciate that the selection of a particular adjuvant might depend on the one or more biologically active agents to be delivered to a subject, the disease or disorder to be treated by the active agent, and the release profile desired for the one or more biologically active agents. Illustrative examples of suitable adjuvants include incomplete Freunds adjuvant (IFA), Adjuvant 65 (containing peanut oil, mannide monooleate and aluminium monostearate), oil emulsions, Ribi adjuvant, the pluronic polyols, polyamines, Avridine, Quil A, saponin, MPL, QS-21, mineral gels, and aluminium salts such as aluminium hydroxide and aluminium phosphate. Other illustrative examples include oil-in-water emulsions such as SAF-1, SAF-0, MF59, Seppic ISA720, and other particulate adjuvants such as ISCOMs and ISCOM matrix.

In an embodiment, the adjuvant is a Toll-like receptor (TLR) agonist. Suitable TLR agonists will be familiar to persons skilled in the art, illustrative examples of which are described in Smith M. et al. (2018; OncoImmunology, 7(12):e1526250). In an embodiment, the TLR agonist is a CpG oligonucleotide (CpG-ODN).

In an embodiment, the CpG-ODN is conjugated to a protein, chemical or peptide molecule prior to loading onto the SPF.

In an embodiment, the adjuvant comprises a pathogen-associated molecular pattern molecule (PAMP) targeting moiety. PAMPs are small molecular motifs associated with groups of pathogens that are recognized by cells of the innate immune system. They are recognized by Toll-like receptors (TLRs) and other pattern recognition receptors (PRRs) in both plants and animals. They activate innate immune responses, protecting the host from infection, by identifying some conserved non-self molecules. For example, bacterial Lipopolysaccharide (LPS), an endotoxin found on the bacterial cell membrane of a bacterium, is considered to be the prototypical PAMP. LPS is specifically recognized by TLR 4, a recognition receptor of the innate immune system. Other illustrative examples of suitable PAMPs include bacterial flagellin (recognized by TLR 5), lipoteichoic acid from Gram positive bacteria, peptidoglycan, and nucleic acid variants normally associated with viruses, such as double-stranded RNA (dsRNA), recognized by TLR 3 or unmethylated CpG motifs, recognized by TLR 9. One or more PAMPs can be used to increase an immune response against an infectious disease.

Also disclosed herein is a vaccine composition comprising short biocompatible polymer fibres (SPF), wherein the SPF comprise poly(D,L-lactide-co-glycolide) (PLGA), an average diameter in the range of from about 15 nm to about 5 μm and an average length in the range of from about 1 μm to about 3 mm; and wherein the SPF are loaded with (i) an immunogen selected from the group consisting on a tumour cell lysate and a cancer-associated antigen; (ii) a cytokine and (iii) an adjuvant. In an embodiment disclosed herein, the vaccine composition is an injectable composition. In an embodiment, the vaccine composition is formulated for administration through a 22-25 gauge needle.

In an embodiment, the immunogen is a tumour cell lysate. In an embodiment, the tumour cell is a glioblastoma tumour cell. In an embodiment, the glioblastoma is glioblastoma multiforme. In an embodiment, the cytokine is granulocyte-macrophage colony-stimulating factor (GM-CSF). In an embodiment, the adjuvant is a CpG oligonucleotide (CpG-ODN).

In an embodiment, the present invention provides a vaccine composition comprising short biocompatible polymer fibres (SPF), wherein the SPF comprise poly(D,L-lactide-co-glycolide)(PLGA), an average diameter in the range of from about 15 nm to about 5 μm and an average length in the range of from about 1 μm to about 3 mm; and wherein the SPF are loaded with (i) a tumour cell lysate and/or a cancer-associated antigen of a glioblastoma; (ii) granulocyte-macrophage colony-stimulating factor (GM-CSF); and (iii) a CpG oligonucleotide (CpG-ODN). In an embodiment, the SPF comprise 1% Resomer® RG 858 S, Poly(D,L-lactide-co-glycolide) and about 0.2% Poly(D,L-lactide-co-glycolide. In an embodiment disclosed herein, the vaccine composition is an injectable composition. In an embodiment, the vaccine composition is formulated for administration through a 22-25 gauge needle.

### Compositions and Methods of Treatment

As noted elsewhere herein, the present inventors have surprisingly found that SPF are a suitable biocompatible delivery vehicle for the sustained delivery of one or more biologically active agents and are able to do so without compromising the integrity of the biologically active agent. The SPF are therefore particularly suitable for the delivery of biologically active agents in vivo. Thus, in an aspect disclosed herein, there is provided a method of delivering a biologically active agent to a subject in need thereof, the method comprising administering to the subject the composition as described herein.

Also disclosed herein is a method of treating or preventing a disease or disorder in a subject in need thereof, the method comprising administering to the subject the composition as described herein.

The term “subject”, as used herein, refers to a mammalian subject for whom treatment or prophylaxis is desired. Illustrative examples of subjects to which the present invention may be directed include primates, especially humans, companion animals such as cats and dogs and the like, working animals such as horses, donkeys and the like, livestock animals such as sheep, cows, goats, pigs and the like, laboratory test animals such as rabbits, mice, rats, guinea pigs, hamsters and the like and captive wild animals such as those in zoos and wildlife parks, deer, dingoes and the like. It is therefore to be understood that the compositions disclosed herein have clinical as well as veterinary applications. In an embodiment, the subject is a human. The term “subject” does not denote a particular age.

Thus, newborn, adolescent, adult and senescent subjects are contemplated herein.

The compositions disclosed herein are also suitable for veterinary applications.

Thus, in particular embodiments, the subject is a livestock animal, such as cattle, sheep or pigs.

The terms “treating”, “treatment” and the like, are also used interchangeably herein to mean relieving, reducing, alleviating, ameliorating or otherwise inhibiting the progression of the disease or disorder in a subject, including one or more symptoms thereof. The terms “treating”, “treatment” and the like are also used interchangeably herein to mean preventing the disease or disorder from occurring or delaying the onset or subsequent progression of the disease or disorder in a subject that may be predisposed to, or at risk of, the disease or disorder, but has not yet been diagnosed as having it. In that context, the terms “treating”, “treatment” and the like are used interchangeably with terms such as “prophylaxis”, “prophylactic” and “preventive”. As used herein, a composition that “treats” a disease or disorder will ideally eliminate the disease or disorder altogether by eliminating its underlying cause so that the disease or disorder does not develop or re-develop. As used herein, a composition that “ameliorates” the disease or disorder does not eliminate the underlying cause of the disease, but reduces the severity of the disease or disorder as measured by any established grading system and/or as measured by an improvement in the subject's well-being, e.g. decrease in pain and/or discomfort.

Also contemplated herein are adjunct therapies for treating the disease or disorder by using one or more additional therapeutic agents. Without being bound by theory or by a particular mode of application, it will generally be understood that the use of a second immunogen, as herein described, can provide an enhance immune response for the treatment of a disease or disorder in the subject.

In some in vivo approaches, the compositions are administered to the subject in a therapeutically effective amount. As used herein, the term “effective amount” or “therapeutically effective amount” means an amount sufficient to relieve, reduce, alleviate, ameliorate or otherwise inhibit the progression of the disease or disorder in a subject and/or one or more symptoms thereof, or to otherwise provide a desired pharmacologic and/or physiologic effect. The precise dosage of the composition will typically depend on the amount of one or more biologically active agents loaded therein, and may also vary according to a variety of additional factors, such as subject-dependent variables (e.g., age, immune system health, etc.), the type and/or severity of the disease or disorder, and the treatment being effected.

For in vivo applications in particular, suitable routes of administration of the compositions disclosed herein will be familiar to persons skilled in the art and will likely to depend on the type, severity and/or location of the disease or disorder to be treated. In an embodiment, the composition is formulated for administration to the subject subcutaneously.

Subcutaneous administration is particularly suited to the compositions comprising an immunogen, where a therapeutically effective immune response towards the immunogen is desired. For instance, upon administration, the SPF will degrade over time to sustainably release the immunogen. This advantageous property of SPF slows the otherwise immediate and rapid diffusion of the immunogen at the site of injection. As the SPF also advantageously protect the immunogen during storage and upon administration, the compositions disclosed herein provide for a more efficient local immune response against the immunogen in vivo. As noted elsewhere herein, the present inventors have unexpectedly found that compositions of SPF can protect the biological activity of the one or more biologically active agents over an extended period of time. By “protect” it is meant that at least some of the biological activity of the one or more biologically active agents that is/are incorporated in the SPF is preserved, such that, upon release from the SPF, the one or more biologically active agents retain a sufficient amount of their biological activity. It is to be understood that it is not necessary for the biologically active agent released from the SPF to retain all (i.e., 100%) of its biological activity (i.e., when compared to the level of biological activity prior to being incorporated into the SPF) and that it is sufficient that the biological agent retains at least some of its biological activity to the extent that it is capable of exerting its biological activity upon release. In an embodiment, the one or more biologically active agents retain, upon release from the SPF, at least 10% of their biological activity (e.g., 10%, 15%, 20%, 25%, 30%, 35%, 40%, 45%, 50%, 55%, 60%, 65%, 70%, 75%, 80%, 85%, 90%, 95% or 100%) when compared to their activity prior to being incorporated into the SPF. In an embodiment, the one or more biologically active agents retain, upon release from the SPF, at least 10% of their biological activity, preferably at least 20% of their biological activity, preferably at least 30% of their biological activity, preferably at least 40% of their biological activity, preferably at least 50% of their biological activity, preferably at least 60% of their biological activity, preferably at least 70% of their biological activity, preferably at least 80% of their biological activity or more preferably at least 90% of their biological activity when compared to their activity prior to being incorporated into the SPF.

The present inventors have surprisingly shown that the biological activity of agents incorporated into the SPF is retained over a period of time of at least 3 to 28 days (e.g., 3 days, 4 days, 5 days, 6 days, 7 days, 8 days, 9 days, 10 days, 11 days and so on). In an embodiment disclosed herein, the one or more biologically active agents retain their biological activity over a period of time of at least 3 days, preferably over a period of at least 7 days, preferably over a period of at least 14 days, preferably over a period of at least 21 hours, or more preferably over a period of at least 28 days when incorporated in the SPF. In an embodiment, the composition is formulated for intramuscular administration to the subject. In an embodiment, the composition is formulated for transdermal administration to the subject.

In an embodiment, the composition is administered to the subject subcutaneously.

It is to be understood that the compositions disclosed herein are suitable for the treatment of any disease or disorder that is treatable by the administration of one or more biologically active agents, in particular where such treatment is responsive to the sustained release of the one or more biologically active agents upon administration in vivo. Diseases and disorders that can be treated by the administration of the compositions disclosed herein will be familiar to persons skilled in the art. Illustrative examples of which include cancer, virus infection, bacterial infection and autoimmune diseases. In an embodiment, the disease or disorder is cancer. Illustrative examples of the type of cancers that may be treated by the compositions disclosed herein will be familiar to persons skilled in the art, illustrative examples of which include leukemias, seminomas, melanomas, teratomas, lymphomas, neuroblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENI) cancer, breast cancer, prostate cancer, cancer of the uterus, ovarian cancer, and lung cancer, lung carcinomas, prostate carcinomas, colon carcinomas, renal cell carcinomas, cervical carcinomas and the metastases thereof. In an embodiment, the cancer is a glioblastoma. In an embodiment, the cancer is glioblastoma multiforme.

In a preferred embodiment, the compositions disclosed herein are used as part of a vaccine strategy. For example, the compositions can be used to deliver an antigen, an immunostimulant, an adjuvant, or a combination thereof. In some embodiments, the compositions include a target moiety that directs the delivery vehicle to specific immune cells, for example, antigen presenting cells such as dendritic cells. In some embodiments, the compositions include one or more antigen presenting cell targeting moieties displayed on the outer shell, and TLR ligands, alone or in combination with an antigen/immunogen.

The antigen/immunogen can be any known antigen/immunogen, for example, an antigen derived from a bacteria, a virus, a fungi, a parasite, or another microbe, environmental antigens or cancer-associated antigens, as described elsewhere herein.

In another aspect, there is provided a composition as described herein for use in the delivery of the one or more biologically active agents to a subject in need thereof.

In another aspect, there is provided a composition as described herein for use in the treatment or prevention of a disease or disorder when administered to a subject in need thereof.

In another aspect, there is provided use of the composition as described herein in the manufacture of a medicament for the treatment or prevention of a disease or disorder in a subject in need thereof.

Non-limiting examples of other diseases that can be treated using the compositions and methods disclosed herein include infectious diseases, viral or microbial, in which a combination antiviral or antibiotic regimen, respectively, is the desirable strategy. For example, an anti-HIV formulation could include activators to initiate HIV replication, inhibitors that prevent HIV infection of new cells and a mixture of death-inducers that are exclusively activated within the infected cell with no harm befalling the others. The SPF can be fabricated with an antibody (or an antigen-binding fragment thereof) that attaches specifically to a molecule expressed on all human T-cells. This serves as the targeting vehicle that protects the encased components and fuses with target T-cells.

The compositions disclosed herein can be used to deliver an effective amount of one or more therapeutic, diagnostic, and/or prophylactic agents to an individual in need of such treatment. The amount of the one or more biologically active agents to be delivered to the subject in need thereof can be readily determine by the prescribing physician and is likely to be dependent on subject-dependent variables such as age, weight and the nature and/or severity of the disease or disorder to be treated.

The compositions are also useful in drug delivery (as used herein “drug” includes therapeutic, nutritional, diagnostic and prophylactic agents), whether injected intravenously, subcutaneously, transdermally (e.g., via a transdermal patch) or intramuscularly, administered to the nasal or pulmonary system, injected into a tumour milieu, administered to a mucosal surface (vaginal, rectal, buccal, sublingual), or encapsulated for oral delivery.

The compositions can also be used for cell transfection of polynucleotides. As discussed below, transfection can occur in vitro or in vivo, and can be employed in a variety of applications, including gene therapy and disease treatment.

Suitable polynucleotides that can be delivered by the compositions disclosed herein can readily be determined by persons skilled in the art depending on the disease or disorder to be treated and in some instances will encode a biological and/or therapeutic agent, such as an immunogen, including those described elsewhere wherein. The polynucleotide can be a gene or cDNA of interest, a functional nucleic acid molecule such as an inhibitory RNA, a tRNA, an rRNA, an siRNA, an shRNA, an mRNA or a guide RNA or an expression vector encoding a gene or cDNA of interest, a functional nucleic acid a tRNA, an rRNA, an siRNA, an shRNA, an mRNA or a guide RNA. In some embodiments, the polynucleotide comprises a functional group. Suitable functional groups will be familiar to persons skilled in the art, an illustrative example of which includes a detectable moiety (e.g., a fluorescent marker/dye, a radioisotope, biotin, streptavidin, etc.).

In some embodiments, the polynucleotide is not integrated into the host cell's genome, but rather remains extrachromosomal. Such embodiments can be useful for transient or regulated expression of a polynucleotide, and may reduce the risk of insertional mutagenesis.

In some embodiments, the polynucleotide is integrated into the host cell's genome. For example, gene therapy is a technique for correcting defective genes responsible for disease development. Researchers may use one of several approaches for correcting faulty genes. For example, (a) a normal gene can be inserted into a non-specific location within the genome to replace a non-functional gene; (b) an abnormal gene can be swapped for a normal gene through homologous recombination; (c) an abnormal gene can be repaired through selective reverse mutation, with a view to returning the gene to its normal function; or (d) the regulation (the degree to which a gene is turned on or oft) of a particular gene can be altered.

Gene therapy can include the use of viral vectors, for example, adenovirus, adeno-associated virus, herpes virus, vaccinia virus, polio virus, AIDS virus, neuronal trophic virus, Sindbis and other RNA viruses, including these viruses with the HIV backbone. Also useful are any viral families which share the properties of these viruses which make them suitable for use as vectors. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA.

Gene targeting via target recombination, such as homologous recombination (HR), is another strategy for gene correction. Gene correction at a target locus can be mediated by donor DNA fragments homologous to the target gene. One method of targeted recombination includes the use of triplex-forming oligonucleotides (TFOs) which bind as third strands to homopurine/homopyrimidine sites in duplex DNA in a sequence-specific manner. Triplex forming oligonucleotides can interact with either double-stranded or single-stranded nucleic acids. Suitable methods for targeted gene therapy using triplex-forming oligonucleotides (TFO's) and peptide nucleic acids (PNAs) will be familiar to persons skilled in the art, such as those described in US 2007-0219122 and US 2008-050920.

Double duplex-forming molecules, such as a pair of pseudocomplementary oligonucleotides, can also induce recombination with a donor oligonucleotide at a chromosomal site. Use of pseudocomplementary oligonucleotides in targeted gene therapy is described in US 2011-0262406. Pseudocomplementary oligonucleotides are complementary oligonucleotides that contain one or more modifications such that they do not recognize or hybridize to each other, for example due to steric hindrance, but each can recognize and hybridize to complementary nucleic acid strands at the target site. In some embodiments, pseudocomplementary oligonucleotides are pseudocomplementary peptide nucleic acids (pcPNAs). Pseudocomplementary oligonucleotides can be more efficient and provide increased target site flexibility over methods of induced recombination such as triple-helix oligonucleotides and bis-peptide nucleic acids which require a polypurine sequence in the target double-stranded DNA.

As noted elsewhere herein, regimes requiring an initial injection to be followed up by one or more subsequent injections or booster injections may be simplified, as the extended bioavailability provided by the compositions disclosed herein means that an effective physiological benefits may be achieved with a single injection, thus obviating the need for subsequent or booster injections to be administered. For instance, compositions disclosed herein comprising an immunogen may induce effective protective immunity (i.e., antibody levels) in a subject following a single injection without the need for subsequent follow up single or multiple injections. An effective level of immunity can be maintained over a number of weeks. In some embodiments, an effective level of immunity could be maintained over a period of several months, and in one embodiment, immunity may be maintained for more than a year. The ability to reduce the number of injections may therefore afford increased convenience to a subject receiving the injections, as well as cost savings to the manufacturer and the consumer.

In use, the composition may be contained in a syringe chamber and injected through the lumen of a needle for administration to a subject. In an embodiment, the composition can be suitably administered via a gauge 23 needle.

In still a further aspect, the invention provides a method of delivering a biologically active agent to a subject comprising the step of administering a composition as described herein to the subject by injection.

The present disclosure also extends to compositions comprising one or more heterogeneous subsets of loaded SPF, wherein each subset of loaded SPF comprises a different biological active agent. For example, the compositions described herein may comprise a first SPF loaded with a first biologically active agent and a second SPF loaded with a second biologically active agent, wherein the first biologically active agent is different to the second biologically active agent. This may be desirable where the nature of the first and second biologically active agents cannot be incorporated (or efficiently incorporated) into the SP together, as described herein, because of the nature of the first and second biologically active agents (e.g., their structure, concentration, solubility, ionic strength, etc.). Thus, where it is desirable to incorporate a first biologically active agent and a second or subsequent biologically active agent into a composition, as described herein, the first biologically active agent can be incorporated into a first subset of SPF and the second or subsequent biologically active agents can be incorporated into a second or subsequent subset of SPF, and the first and second and/or subsequent subsets of loaded SPF combined to form the composition described herein. Alternatively, the first biologically active agent can be incorporated into a first subset of SPF and the second and/or subsequent biologically active agents can be incorporated into a second and/or subsequent subset of SPF, and the first and second and/or subsequent subsets of loaded SPF separately formulated for sequential administration to a subject in need thereof.

### Manufacturing Processes

In an embodiment of the present invention, there is provided a process for the preparation of a composition for rapid and sustained delivery of one or more biologically active agents, the process comprising:

(a) introducing a stream of biocompatible polymer fibre-forming liquid into a dispersion medium having a viscosity in the range of from about 1 to 100 centiPoise (cP);
(b) forming a filament in the dispersion medium from the stream of the fibre-forming liquid of (a);
(c) shearing the filament of (b) under conditions allowing fragmentation of the filament and formation of short biocompatible polymer fibres (SPF), wherein the SPF have an average length in the range of from about 1 μm to about 3 mm, and an average diameter in the range of from about 15 nm to about 5 μm; and
(d) loading the SPF of (c) with one or more biologically active agents;
thereby producing a composition for the rapid and sustained delivery of one or more biologically active agents.

In another aspect disclosed herein, there is provided a process for the preparation of a composition for the sustained release of one or more biologically active agents, the process comprising:

(a) providing a mixture comprising (i) a biodegradable polymer fibre-forming liquid and (ii) one or more biologically active agents;
(b) introducing a stream of the mixture of (a) into a dispersion medium having a viscosity in the range of from about 1 to 100 centiPoise (cP);
(b) forming a filament in the dispersion medium from the stream of (a);
(c) shearing the filament of (b) under conditions allowing fragmentation of the filament and formation of short biocompatible polymer fibres (SPF), wherein the SPF have an average length in the range of from about 1 μm to about 3 mm, and an average diameter in the range of from about 15 nm to about 5 μm.

Alternatively, or in addition, the one or more biologically active agents may be incorporated (i.e, loaded) into the SPF subsequent to the formation of the SPF. For example, the SPF, as described herein, once formed, may be combined with the one or more biologically active agents under conditions and for a period of time sufficient to allow the one or more biologically active agents to be incorporated into the SPF so as to form the loaded SPF. Thus, in an embodiment disclosed herein, there is provided a process for the preparation of a composition for rapid and sustained delivery of one or more biologically active agents, the process comprising:

(a) providing short biocompatible polymer fibres (SPF), as described herein; and
(b) exposing the SPF to one or more biologically active agents, as described herein, under conditions and for a period of time sufficient to allow the one or more biologically active agents to be incorporated into the SPF so as to form SPF loaded with the one or more biologically active agents.

In some embodiments, step (b) may be repeated, as necessary, to increase the rate of incorporation of the one or more biologically active agents into the loaded SPF. Alternatively, or in addition, the process may further comprise: (c) repeating step (b) by exposing the loaded SPF to one or more additional biologically active agents, wherein the one or more additional biologically active agents are different to the one or more biologically active agents of step (c), thereby forming SPF loaded with two or more different biologically active agents. This may be advantageous, for example, in vaccine compositions, where it desirable to use a single composition of loaded SPF to raise an immune response against multiple immunogens.

Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

The invention will now be described with reference to the following Examples which illustrate some preferred aspects of the present invention. However, it is to be understood that the particularity of the following description of the invention is not to supersede the generality of the preceding description of the invention and that various other modifications and/or alterations may be made without departing from the spirit of the present invention, as disclosed herein.

### EXAMPLES

### Example 1—Manufacture of Short Biocompatible Polymer Fibres (SPF)

SPF were manufactured by a modification to the methods previously described in WO 2013/056312, the contents of which are incorporated herein by reference in their entirety.

Briefly, poly(D,L-lactide-co-glycolide) (ester terminated; molecular weight 50-75 kDa) (PLGA1) was mixed in DMSO solutions at varying concentrations (0.234, 0.47 and 0.94% w/v) with 1% w/v Resomer® RG 858 S (Poly(D,L-lactide-co-glycolide) ester terminated, lactide:glycolide 85:15, Mw 190-240 kDa), or used alone in DMSO (1.88 and 3.75% w/v). Resomer alone (1, 2 and 4% w/v in DMSO) was also used. 5% w/v Tween 80 was added to 1-butanol (wash solution) as a surfactant to prevent PSF aggregation. Various coagulating fluids were investigated (ethanol, 80% v ethanol 20% v 1-butanol). Different needle sizes (23 G and 25 G) were trialed along with a variety of wash protocols.

The optimal protocol for the manufacture of SPF for use in the following studies comprised 1% w/v Resomer with 0.234% w/v PLGA1 using 1-butanol as the gelating fluid and 25 G needle. Washes consisted of 5% w/v Tween80 in butanol, 5% w/v Tween80 in 80% w/v ethanol, 5% w/v Tween80 in saline, 2% w/v Tween80 in saline (twice), saline (three times). The polymer fibres made by this process will be referred to interchangeably herein below as PLGA-SPF or SPF.

SPF were consistently non-uniform in shape and small in size, with an average length in the range of from about 1 μm to about 3 mm and an average diameter in the range of from about 15 nm to about 5 μm. SPF were frozen at −80° C. without undergoing significant change to size or resuspension properties. Subsequent analysis showed that the synthesised SPF were sterile.

### Example 2—Incorporation and Retention of Activity of Biological Material

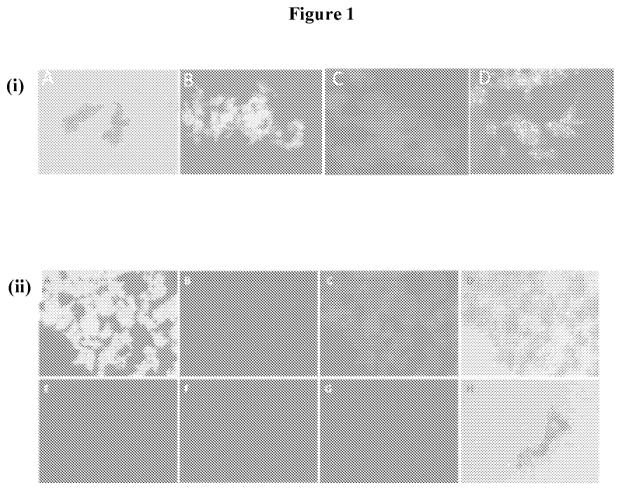

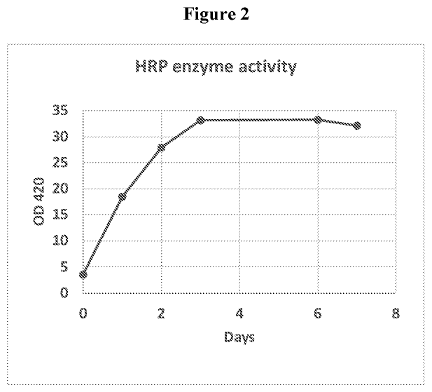

Large proteins (14-100 kDa), peptides (1 kDa) and ssDNA (22-mers) tagged with fluorescent markers were successfully incorporated into SPF as demonstrated using fluorescent microscopy. These were either incorporated singularly (FIG. 1(i)) or together (FIG. 1(ii)) into SPF with equal success. (FIG. 1). Subsequent analysis showed an incorporation rate in this instance of about 37%. However, as noted elsewhere herein, the loading of the biological material to the SPF may vary depending on, for example, the solubility of the SPF and the biological material to be loaded (e.g., size, net charge, molecular weight). Enzyme incorporation into SPF with retention of function was demonstrated by using horse radish peroxidase (HRP). HRP-SPF was assayed by incubating in water for 1, 2, 3, and 6 days with aliquots taken at each time point and stored at −80° C. Activity of released HRP was measured by colorimetric assay and showed HRP was enzymatically active up to 6 days (FIG. 2).

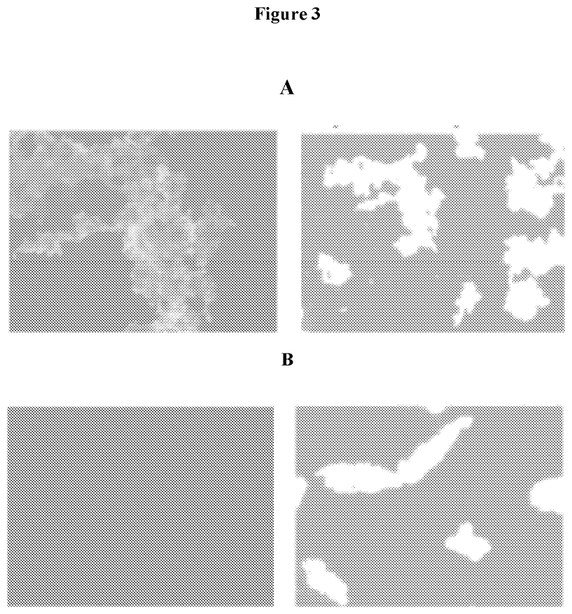

Fluorescent tags are not ideal in therapeutic products, therefore to eliminate the possibility that the fluorescent tags used in these studies had an effect on incorporation untagged protein were tested for incorporation to eliminate effects of the tag during incorporation. Ovalbumin (OVA) is frequently used as a model antigen in animal experiments including the mouse GBM tumour model that uses the OVA expressing GL261-Qaud cell line as a tumour surrogate. OVA was incorporated into PLGA with the use of 70% DMSO due to poor DMSO solubility. Manufacture protocol was modified to exclude the overnight incubation which reduced the amount of premature coagulation of PLGA and OVA which occurred with overnight incubation. Following SPF formation, OVA incorporation was detected using immunofluorescence techniques, using an OVA antibody and a fluorescent 488 secondary antibody, (FIG. 3).

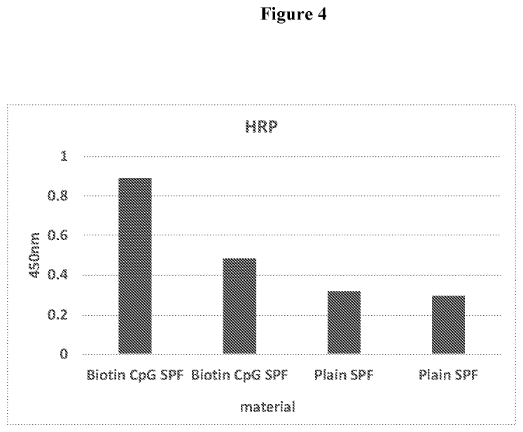

The removal of the fluorescent tag from DNA affected the rate of incorporation into the SPF, which was predicted to be due to the insolubility of DNA in DMSO. Biotinylation of DNA by linking biotin to DNA at the 3′ end overcame this problem. Biotin is TGA approved for medical use. The biotin-DNA complex was successfully incorporated into PLGA and measured by detection of biotin within the SPF using an anti-biotin antibody HRP assay (FIG. 4).

### Example 3—Incorporation and Release Rates of Biological Material

Incorporation rates were measured by adding a known amount of protein to the manufacture of SPF and redissolving in a known amount of DMSO. Released protein was determined by protein estimates using a spectrophotometer at OD280. This method showed 40-50% of biological agents were successfully incorporated and released during PLGA-SPF manufacture.

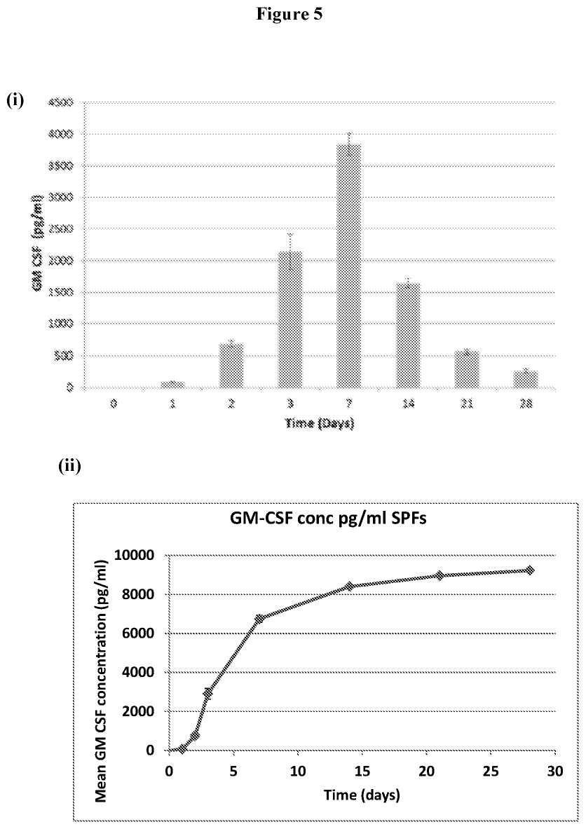

Release studies using ELISA assay combined with biological assays showed that biological activity of the incorporated components was retained upon release from the SPF. Recombinant human granulocyte macrophage colony-stimulating factor (GMCSF), a cytokine that plays a role in the modulation of the immune response and dendritic cell differentiation, and a component of the immunotherapy cocktail of drugs, was incorporated into PLGA-SPF, washed and kinetics of release was studied. GM-CSF-loaded SPF were incubated in saline for 0, 1, 2, 3, 7, 14, 21 and 28 days and an ELISA assay was used to measure GM-CSF release. Results showed that incorporated GM-CSF was continuously released over a 28 day period, with the greatest of release occurring in the first 3 days (7 ng/ml), followed by sustained slow release at decreasing concentrations; 4 ng/ml at 7 days, 2 ng/ml at 14 days, 1.8 ng/ml at 21 days and 0.4 ng/ml at 28 days (FIG. 5).

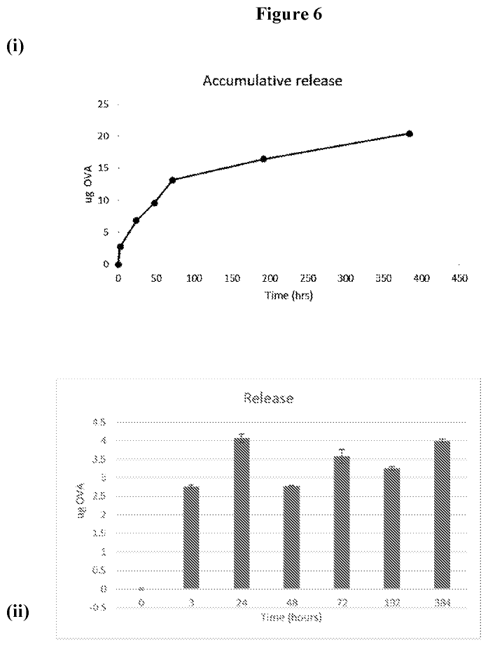

Similar assays were conducted with OVA in PLGA over a 16 day release period, and after 2 hours alongside GMCSF release for comparison (FIG. 6).

### Example 4—Toxicity and Other Effects on Cells In Vitro

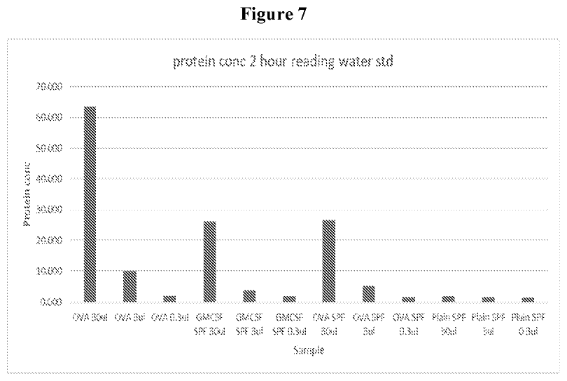

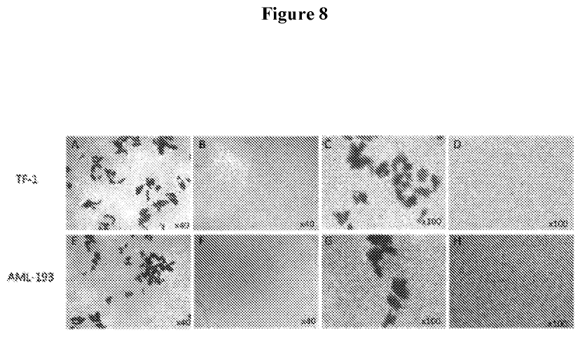

Toxicity of soluble PLGA, SPF and 0.5% DMSO was determined by adding these components to cells in culture. AML-193 and TH-1 (leukaemia cell lines) showed no inhibition of growth or cell death after being exposed PLGA and SPF for 3 days in culture (FIG. 7). Cell morphology was not affected by the presence of SPF in culture and SPF did not cause repulsion/aggregation of cells, suggesting that SPF are inert to cells under these culture conditions (FIG. 8).

### Example 5—Retention of Biological Activity in Cell Culture

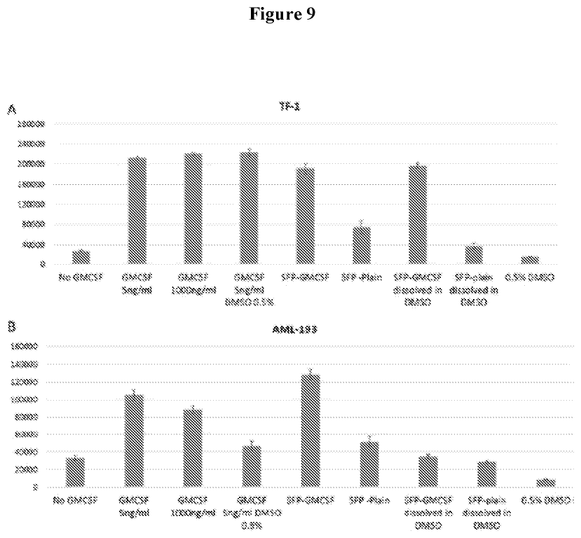

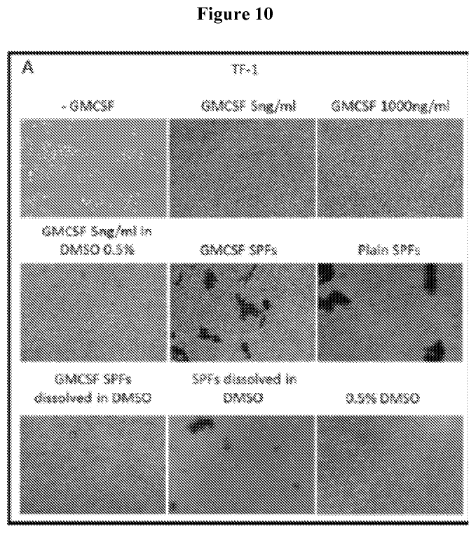

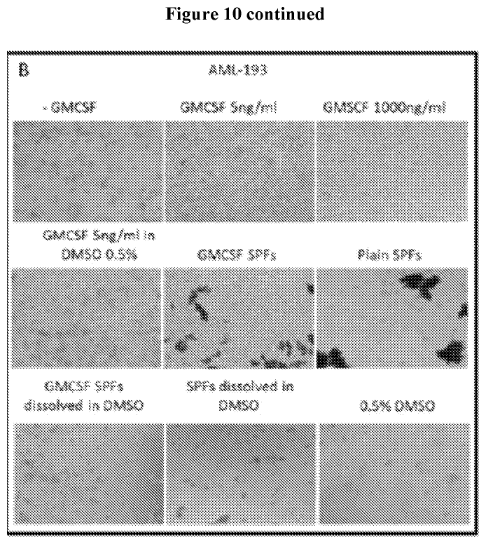

The biological activity of GM-CSF-loaded SPF was tested in vitro by using GM-CSF-sensitive leukaemia cell lines—AML-193 and TF-1. These cells failed to grow in the absence of GM-SCF. Cells were starved of GM-CSF for 24 hours prior to treatment. SPF loaded with GMCSF and plain (empty) SPF were incubated with cells for 4 days. SPF dissolved in DMSO to release GM-CSF were also tested. Both AML-193 and TF-1 grew in the presence of whole and dissolved GM-CSF-loaded SPF, but failed to grow in the presence of empty or dissolved SPF (FIGS. 9 and 10).

### Example 6—SPF Protection of Biological Activity

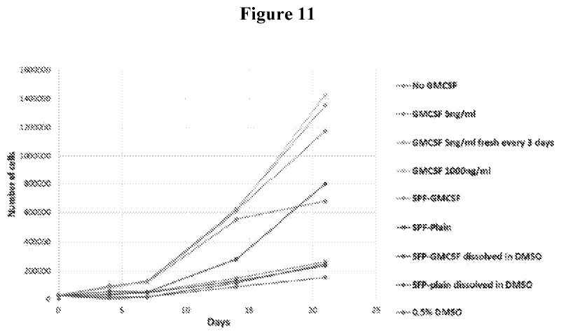

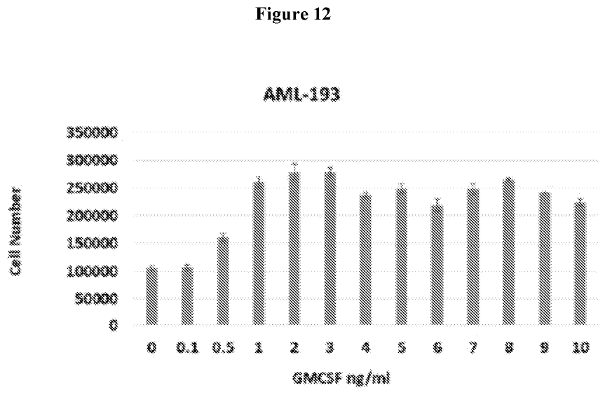

In order to determine how long SPF could actively deliver functional GM-CSF to the cells, a time course experiment was performed. AML-193 cells were incubated under the same condition for 4, 7, 14 and 21 days (FIG. 11). GM-CSF added at the beginning of culture lost biological activity after day 14, while cells incubated in the presence of GM-CSF-loaded SPF continued to grow for 21 days, similar to the rate of growth that was seen in cells incubated with fresh GM-CSF (5 ng/ml) added every 3 days, or cells incubated with a dose GM-CSF at 1000 mg/ml. When GM-CSF-loaded SPF were dissolved in DMSO, the GM-CSF released from the SPF could only sustain growth for 7 days, consistent with the expected half-life of GM-CSF. These data suggest that, when GM-CSF was incorporated into the SPF, its biological activity was protected and that the GM-CSF remained biologically active until release from the SPF. Dose requirement for AML-193 cells suggests that GM-CSF was released at a minimum concentration of >0.5 ng/ml (sensitivity range of cells) (FIG. 12).

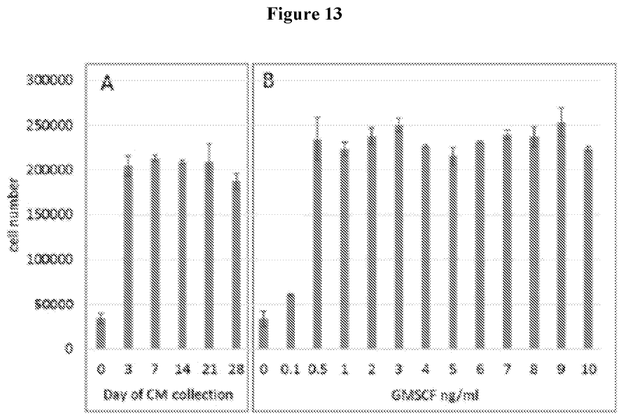

To further validate the protective role of SPF for biologically active agents, GM-CSF-loaded SPF were incubated in media and samples were collected at regular intervals over 28 days. Media was collected and replaced at day 3, 7, 14, 21 and 28, ensuring only freshly released GM-CSF was within each time point, and then added to THP-1 GM-CSF-sensitive cells and assayed for cell growth (FIG. 13a). Cells grew in media collected from all time points suggesting that media remained biologically active at or above 0.1 ng/ml as determined by the GM-CSF dose response of TF-1 cells (FIG. 13b).

### Example 7—Maintenance of Complex Biological Functions for Immunotherapy

Immunotherapy typically requires a number of steps to program the immune system. In an illustrative example, one of the steps involves the addition of GMSCF which acts to differentiate monocytes to yield dendritic cells (DCs). Another of these steps involves the addition of CpG, a DNA sequence designed to mimic bacteria in order to alert the immune cells to attack. The third step involves programming the DCs to signal to T cells which then kill tumour cells.

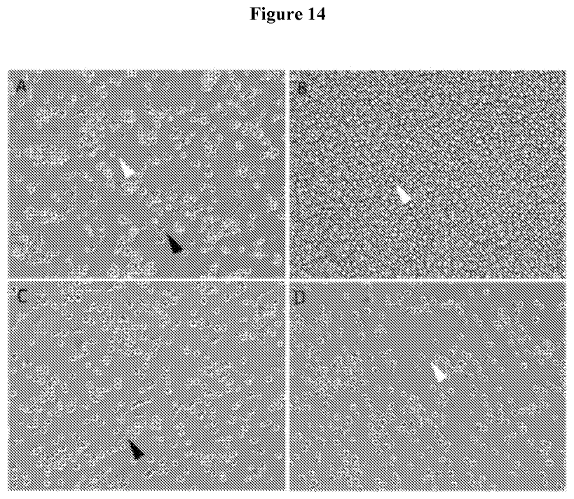

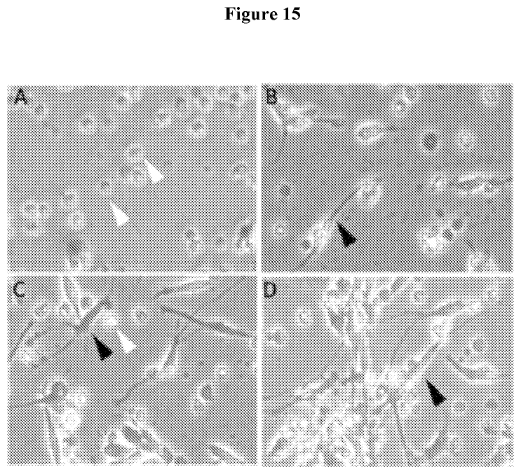

The first two steps were tested for use of SPF mediated immunotherapy using human primary monocytes. GM-CSF (a cytokine that drives the differentiation of monocytes into dendritic cells; DCs) and CpG-ODN (a TLR agonist) were tested for their use in SPF-mediated immunotherapy using THP-1 monocytic cell line and human primary monocytes. GM-CSF-loaded SPF, with or without CpG-ODN, were manufactured and incubated for 2 days in cell culture media. The media was then collected and used to assess monocyte differentiation (as determined by cell morphology), with the results compared to GM-CSF alone, CpG-ODN alone and plain (empty) SPF. Monocytic cells in culture typically appear round, while DCs are typically elongated. Media from GM-CSF-loaded SPF was shown to differentiate THP-1 cells towards a DC lineage, similar to the effect seen when cells were cultured in the presence of GM-CSF alone. By contrast, empty SPF failed to induce cell differentiate (FIG. 14). Similarly, human monocytes were showed to differentiate towards a DC linage in the presence of GM-CSF-CpG-ODN-loaded SPF and GM-CSF-loaded SPF when compared to empty SPF (FIG. 15).

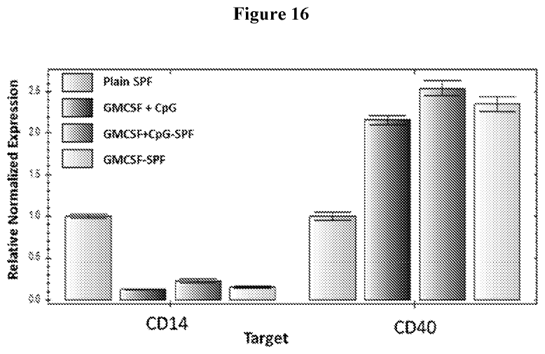

DC biomarkers CD14 and CD40 were used to monitor differentiation of human monocytes towards a DC lineage. RNA was collected from the cells following differentiation and analysed for the expression of these biomarkers by quantitative polymerase chain reaction (qPCR) (FIG. 16). Monocytic cells show decreased CD14 expression upon differentiation into DCs, while CD40 expression is increased. Consistent with the observed morphological changes, the gene expression changes for CD14 and CD40 were also indicative of differentiation of the monocytes towards a DC lineage when the cells were cultured in the presence of GM-CSF-loaded and GM-CSF-CpG-ODN-loaded SPF.

### Example 8—Maintenance of Complex Biological Functions for Immunotherapy In Vivo

SPF has been validated as a proof of concept for delivery of biological materials. This involves incorporation, protection of biological activity and release. SPF has been trialed as a carrier for immunotherapy biological agents and shown that two of the steps required for the activation of the immune system were delivered successfully. Importantly, SPF protected and slowly released the vaccine components to allow prolonged exposure to the immune system. We have also shown that SPF loaded with GMSCF can differentiate the human monocytes to yield morphologically defined dendritic cells, confirming activity in culture.

Mouse in vivo experiments investigated tolerance and generation of cytotoxic T lymphocytes against a model antigen (OVA). Deakin University animal ethics approval was granted for this study.

### Treatment Groups of Mice:

Group 1—Functionalised SPF (mouse GM-CSF, CpG ODN 2395, OVA)
Group 2—Plain SPF (negative control)
Group 3—Saline (negative control)
Group 4—Drug alone (positive control)
Functionalised SPF vaccine was injected subcutaneously into the neck scruff of C57BL/6J immunocompetent mice. On day 21 after injection the mice were humanely killed and blood and spleen of the animal was collected for analysis.

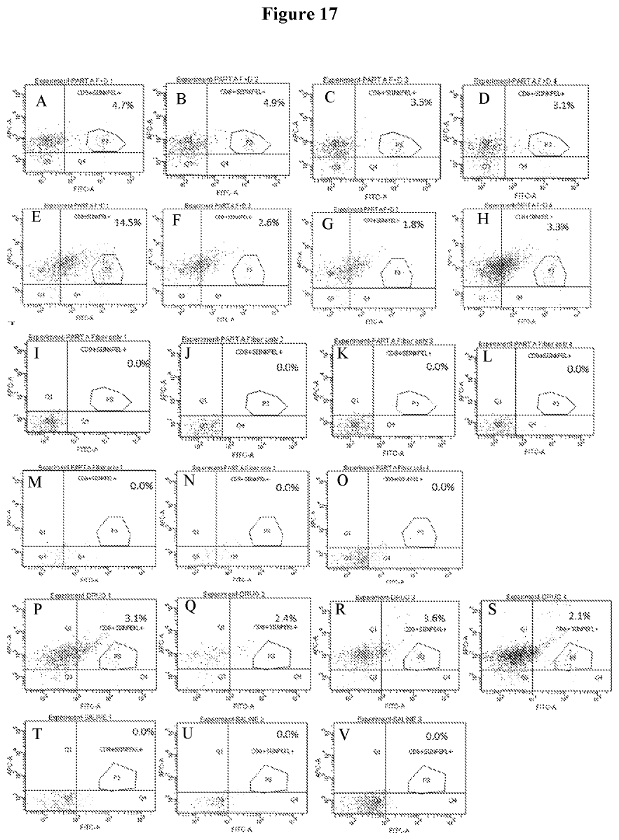

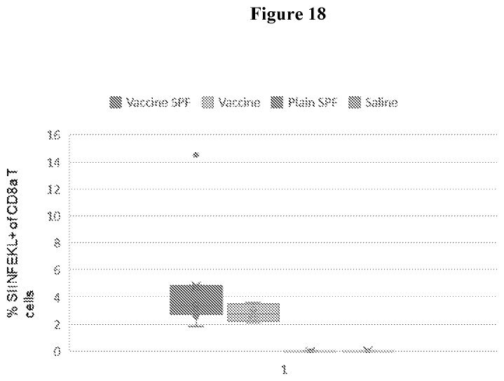

Red blood cells were lysed using lysis buffer to yield a population of leukocytes. Half of the remaining cells were first stained with fluorescently labelled anti-mouse H-2Kb bound to SIINFEKL antibody, which is an antibody that specifically detects T cells activated by ovalbumin antigen. The cells were then stained with a fluorescently labelled CD8+ antibody stain to detect all cytotoxic T cells. CD8+ T cells protect against infection from intracellular bacteria and parasites by lysing infected targets. Most cytotoxic T cells express T-cell receptors (TCRs) that can recognize a specific antigen. Flow cytometry analysis was carried out to calculate the percentage of T cells can that recognize OVA (antigenic sequence SIINFEKL within OVA) in the control and treatment groups (FIG. 17). Data showed that mice administered functionalised SPF vaccine (mouse GM-CSF, CpG ODN 2395, OVA) had higher levels of CD8+ T cells with the SIINFEKL surface recognition marker (FIG. 18).

Interferon gamma (IFNγ) is a key moderator of cell-mediated immunity with diverse, mainly pro-inflammatory actions on immunocytes and target tissue. Recent studies have shown it may enhance anti-tumor and antiviral effects of CD8+ T cells. The ability of CD8+ T cells to produce IFNγ enhanced their ability to migrate to the site of antigen-presenting cells. It markedly increases T cell-mediated killing by upregulating MHC-I expression on target cells, and may promote target cell differentiation and death directly. Antigen-specific CD8+ T cells that produce INFγ when exposed to the recognition antigen are therefore predicted to have a better tumour killing capability.

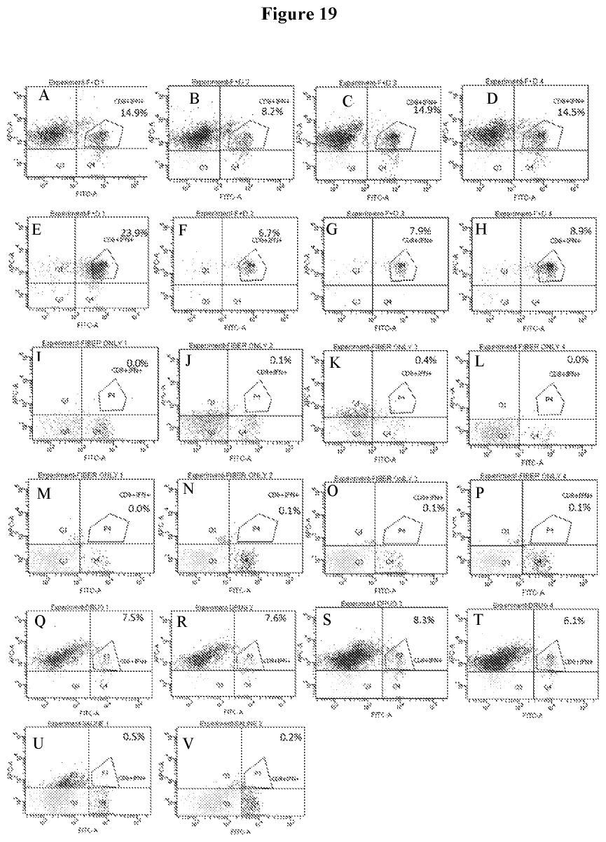

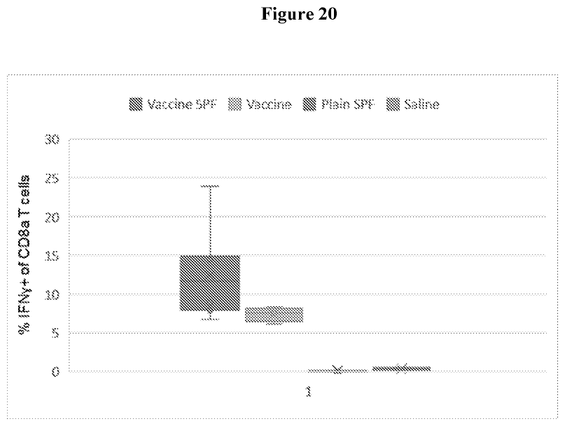

The other half of the collected leukocytes were used to detect IFN-γ producing T cells when they are re-exposed to OVA antigen. Cells were co-incubated with OVA 257-264 peptide (SIINFEKL) which is used to detect a strong CD8+ cytotoxic T cell response. A protein transport inhibitor (GolgiStop) was added before the cells are fluorescently labelled with a CD8+ antibody stain to detect all cytotoxic T cells. The cells were then incubated with a reagent that makes the cell membrane permeable (Cytomix/Cytoperm). They were then stained with a fluorescently labelled IFN-γ stain for flow cytometry analysis (FIG. 19). Data showed that mice administered vaccine via SFP delivery had higher levels of OVA recognising cytotoxic T cells capable of detecting and destroying cells expressing the OVA antigen (FIG. 20).

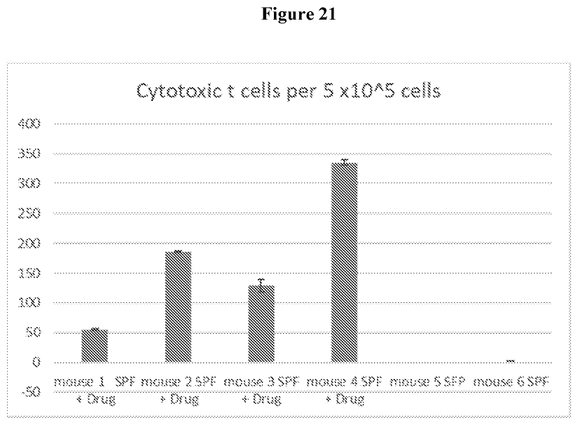

The ability of SINFEKL to induce CD8+ T cell responses in vivo was also determined using an IFNγ ELISpot assay. Spleenocytes were collected from mice spleen at termination of the experiment. Spleenocytes were isolated cutting open the spleen and filtering through a 70 micron sieve. Cells were washed with 10 ml cold RPMI and red blood cells were lysed using 1×RBC Lysis Buffer for 5 mins. The reaction was stopped with cold PBS and cells were centrifuged at 300 g/5 mins and washed with 10 ml cold PBS. A cell count was performed and 5×10⁵ spleenocytes were plated into each Elsispot well with or without SINFEKL. PMA (10 ng/ml) and inomycin (1 mM) (IFNγ activators) were also used as a positive control. Cells were incubated overnight and the production of IFN-γ was determined by ELISpot assay following the manufacturers instruction. Mice injected with SPF containing OVA generated measurable IFN-γ secreting cells to SIINFEKL or while SPF only mice did not (FIG. 21).

### CONCLUSION

The present inventors have, for the first time, validated SPF as a delivery vehicle for the rapid and sustained delivery of biologically active agents and that the SPF are capable of protecting the biologically active agents overtime, thereby preserving biological activity upon release from the SPF. Importantly, these properties and kinetics of SPF as an ideal delivery vehicle can be replicated in an in vivo setting.

1. A composition for sustained delivery of one or more biologically active agents in vivo, the composition comprising:
short biocompatible polymer fibres (SPF) having an average length in the range of from about 1 μm to about 3 mm, and an average diameter in the range of from about 15 nm to about 5 μm, wherein the SPF are loaded with one or more biologically active agents,
wherein, when administered, the composition provides sustained release of the one or more biologically active agents from the SPF.

2. The composition according to claim 1, wherein the SPF have an average diameter in the range of from about 50 nm to about 500 nm.

3. The composition according to claim 1 or claim 2, wherein the SPF have an average length in the range of from about 1 μm to about 20 μm.

4. The composition according to claim 1 or claim 2, wherein the SPF have an average length in the range of from about 2 μm to about 10 μm.

5. The composition according to any one of claims 1 to 4, wherein the biocompatible polymer is selected from the group consisting of polypeptides, alginates, chitosan, starch, collagen, silk fibroin, polyurethanes, polyacrylic acid, polyacrylates, polyacrylamides, polyesters, polyolefins, boronic acid functionalised polymers, polyvinylalcohol, polyallylamine, polyethyleneimine and polyvinyl pyrrolidone), poly(lactic acid), polyether sulfone, inorganic polymers, and a combination of any of foregoing.

6. The composition according to claim 5, wherein the biocompatible polymer comprises poly(lactic acid).

7. The composition according to claim 6, wherein the poly(lactic acid) is poly(lactic-co-glycolic acid) (PLGA).

8. The composition according to any one of claims 1 to 7, wherein the one or more biologically active agents are selected from the group consisting of a hormone, an antimicrobial, an antiviral, a steroid, a chemotherapy drug, a therapeutic antibody or an antigen-binding fragment thereof a cytokine, an immunogen, a nucleic acid molecule, an adjuvant or a combination of any of the foregoing.

9. The composition according to claim 8, wherein the one or more biologically active agents comprises an immunogen.

10. The composition according to claim 9, wherein the immunogen is selected from the group consisting of a tumour cell, a tumour cell lysate, a virus, a viral antigen, a bacteria, a bacteria cell lysate, a cancer-associated antigen, nucleic acid molecules encoding any of the foregoing and a combination of any of the foregoing.

11. The composition according to claim 10, wherein the immunogen is a tumour cell lysate.

12. The composition according to claim 11, wherein the tumour cell is a glioblastoma tumour cell.

13. The composition according to claim 12, wherein the glioblastoma is glioblastoma multiforme.

14. The composition according to any one of claims 1 to 13, wherein the one or more biologically active agents comprises a cytokine.

15. The composition according to claim 14, wherein the cytokine is granulocyte-macrophage colony-stimulating factor (GM-CSF).

16. The composition according to any one of claims 1 to 15, wherein the one or more biologically active agents comprises an adjuvant.

17. The composition according to claim 16, wherein the adjuvant is a Toll-like receptor (TLR) agonist.

18. The composition according to claim 17, wherein the TLR agonist is a bacterial CpG oligonucleotide (CpG-ODN).

19. The composition according to any one of claims 1 to 17, wherein the composition is formulated for subcutaneous, intramuscular or transdermal administration.

20. A method for rapid and sustained delivery of one or more biologically active agent to a subject in need thereof, the method comprising administering to a subject the composition of any one of claims 1 to 19.

21. The method according to claim 10, wherein the composition is administered to the subject subcutaneously.

22. A method of treating or preventing a disease or disorder in a subject in need thereof, the method comprising administering to a subject the composition of any one of claims 1 to 19.

23. The method according to claim 22, wherein the composition is administered to the subject subcutaneously.

24. The method according to claim 22, wherein the composition is administered to the subject intramuscularly.

25. The method according to claim 22, wherein the composition is administered to the subject transdermally.

26. The method according to any one of claims 22 to 25, wherein the disease or disorder is cancer.

27. The method according to claim 26, wherein the cancer is a glioblastoma.

28. The method according to claim 27, wherein the cancer is glioblastoma multiforme.

29. The composition of any one of claims 1 to 19 for use in the delivery of the one or more biologically active agents to a subject in need thereof.

30. The composition according to claim 29, wherein the composition is formulated for subcutaneous administration to the subject.

31. The composition according to claim 29, wherein the composition is formulated for intramuscular administration to the subject.

32. The composition according to claim 29, wherein the composition is formulated for transdermal administration to the subject.

33. The composition of any one of claims 1 to 19 for use in the treatment or prevention of a disease or disorder when administered to a subject in need thereof.

34. The composition according to claim 33, wherein the composition is formulated for intravascular, subcutaneous, transdermal and/or intramuscular administration to the subject.

35. The composition according to claim 33 or claim 34, wherein the disease or disorder is cancer.

36. The composition according to claim 35, wherein the cancer is a glioblastoma.

37. The composition according to claim 36, wherein the cancer is glioblastoma multiforme.

38. Use of the composition of any one of claims 1 to 19 in the manufacture of a medicament for the treatment or prevention of a disease or disorder in a subject in need thereof.

39. Use according to claim 38, wherein the composition is formulated for subcutaneous administration to the subject.

40. Use according to claim 38 or claim 39, wherein the disease or disorder is cancer.

41. Use according to claim 40, wherein the cancer is a glioblastoma.

42. Use according to claim 41, wherein the cancer is glioblastoma multiforme.

43. A process for the preparation of a composition for rapid and sustained delivery of one or more biologically active agents, the process comprising:
(a) introducing a stream of biocompatible polymer fibre-forming liquid into a dispersion medium having a viscosity in the range of from about 1 to 100 centiPoise (cP);
(b) forming a filament in the dispersion medium from the stream of the fibre-forming liquid of (a);
(c) shearing the filament of (b) under conditions allowing fragmentation of the filament and formation of short biocompatible polymer fibres (SPF), wherein the SPF have an average length in the range of from about 1 μm to about 3 mm, and an average diameter in the range of from about 15 nm to about 5 μm; and
(d) loading the SPF of (c) with one or more biologically active agents.

44. A process for the preparation of a composition for the sustained release of one or more biologically active agents, the process comprising:
(a) providing a mixture comprising (i) a biodegradable polymer fibre-forming liquid and (ii) one or more biologically active agents;
(b) introducing a stream of the mixture of (a) into a dispersion medium having a viscosity in the range of from about 1 to 100 centiPoise (cP);
(b) forming a filament in the dispersion medium from the stream of (a);
(c) shearing the filament of (b) under conditions allowing fragmentation of the filament and formation of short biocompatible polymer fibres (SPF), wherein the SPF have an average length in the range of from about 1 μm to about 3 mm, and an average diameter in the range of from about 15 nm to about 5 μm.

45. The process according to claim 43 or claim 44, wherein the SPF have an average diameter in the range of from about 50 nm to about 500 nm.

46. The process according to any one of claims 43 to 45, wherein the SPF have an average length in the range of from about 1 μm to about 20 μm.

47. The process according to claim 46, wherein the SPF have an average length in the range of from about 2 μm to about 10 μm.

48. The process according to any one of claims 43 to 47, wherein the biocompatible polymer is selected from the group consisting of polypeptides, alginates, chitosan, starch, collagen, silk fibroin, polyurethanes, polyacrylic acid, polyacrylates, polyacrylamides, polyesters, polyolefins, boronic acid functionalised polymers, polyvinylalcohol, polyallylamine, polyethyleneimine and polyvinyl pyrrolidone), poly(lactic acid), polyether sulfone, inorganic polymers, and a combination of any of foregoing.

49. The process according to claim 48, wherein the biocompatible polymer comprises poly(lactic acid).

50. The process according to claim 49, wherein the poly(lactic acid) is poly(lactic-co-glycolic acid) (PLGA).

51. The process according to any one of claims 43 to 50, wherein the one or more biologically active agents are selected from the group consisting of a hormone, an antimicrobial, an antiviral, a steroid, a chemotherapy drug, a therapeutic antibody or an antigen-binding fragment thereof a cytokine, an immunogen, a nucleic acid molecule, an adjuvant or a combination of any of the foregoing.

52. The process according to claim 51, wherein the one or more biologically active agents comprises an immunogen.

53. The process according to claim 52, wherein the immunogen is selected from the group consisting of a tumour cell, a tumour cell lysate, a virus, a viral antigen, a bacteria, a bacteria cell lysate, a cancer-associated antigen, nucleic acid molecules encoding any of the foregoing and a combination of any of the foregoing.

54. The process according to claim 53, wherein the immunogen is a tumour cell lysate.

55. The process according to claim 54, wherein the tumour cell is a glioblastoma tumour cell.

56. The process according to claim 55, wherein the glioblastoma is glioblastoma multiforme.

57. The process according to any one of claims 43 to 56, wherein the one or more biologically active agents comprises a cytokine.

58. The process according to claim 57, wherein the cytokine is granulocyte-macrophage colony-stimulating factor (GM-CSF).

59. The process according to any one of claims 43 to 58, wherein the one or more biologically active agents comprises an adjuvant.

60. The process according to claim 59, wherein the adjuvant is a TLT agonist.

61. The process according to claim 60, wherein the TLR agonist is a CpG oligonucleotide (CpG-ODN).

62. A composition prepared by the process according to any one of claims 43 to 61.

63. A vaccine composition comprising:
short biocompatible polymer fibres (SPF), wherein the SPF comprise poly(D,L-lactide-co-glycolide) (PLGA) and have an average diameter in the range of from about 15 nm to about 5 μm and an average length in the range of from about 1 μm to about 3 mm; and
wherein the SPF are loaded with (i) an immunogen selected from the group consisting of a tumour cell lysate and a cancer-associated antigen; (ii) a cytokine and (iii) an adjuvant.

64. The composition according to claim 63, wherein the immunogen is a tumour cell lysate.

65. The composition according to claim 64, wherein the tumour cell is a glioblastoma tumour cell.

66. The composition according to claim 64, wherein the glioblastoma is glioblastoma multiforme.

67. The composition according to any one of claims 62 to 66, wherein the cytokine is granulocyte-macrophage colony-stimulating factor (GM-CSF).

68. The composition according to any one of claims 62 to 67, wherein the adjuvant is a CpG oligonucleotide (CpG-ODN).

69. A vaccine composition comprising:
short biocompatible polymer fibres (SPF), wherein the SPF comprise poly(D,L-lactide-co-glycolide) (PLGA) and have an average diameter in the range of from about 15 nm to about 5 μm and an average length in the range of from about 1 μm to about 3 mm; and
wherein the SPF are loaded with (i) a tumour cell lysate and/or a cancer-associated antigen of a glioblastoma; (ii) granulocyte-macrophage colony-stimulating factor (GM-CSF); and (iii) a CpG oligonucleotide (CpG-ODN).

70. The composition according to any one of claims 62 to 69, wherein the composition is formulated for subcutaneous, intramuscular or transdermal administration.
